(19) United States
(12) Patent Application Publication
(10) Pub. No.: US 2013/0344055 A1
(43) Pub. Date: Dec. 26, 2013

(54) ANTIMICROBIAL AGENTS

(71) Applicants: Yves Briers, Rohr (CH); Rob Lavigne, Merksem (BE); Maarten Walmagh, Herk-de-Stad (BE); Stefan Miller, Regensburg (DE)

(72) Inventors: Yves Briers, Rohr (CH); Rob Lavigne, Merksem (BE); Maarten Walmagh, Herk-de-Stad (BE); Stefan Miller, Regensburg (DE)

(73) Assignees: KATHOLIEKE UNIVERSITEIT LEUVEN, Leuven (BE); LYSANDO AG, Triesenberg (LI)

(21) Appl. No.: 13/997,058

(22) PCT Filed: Dec. 23, 2011
(86) PCT No.: PCT/EP2011/073908

§ 371 Date: Sep. 3, 2013

(30) Foreign Application Priority Data
Dec. 23, 2010 (EP) 10196890.7

(51) Int. Cl. A01N 37/46; C12Q 1/37; C12N 9/54; C12N 9/52

(52) U.S. Cl. CPC A01N 37/46; C12N 9/52; C12Q 1/37; C12N 9/54
USPC 424/94.63; 435/220; 536/23.2; 435/320.1; 435/252.3; 435/254.2; 435/23; 435/221

(57) ABSTRACT

The present invention relates to a fusion protein composed of an enzyme having the activity of degrading the cell wall of Gram-negative bacteria and/or Gram-positive bacteria and at least two peptide stretches fused to the enzyme at the N- or C-terminus, wherein the peptide stretches are distinct and selected from the group of synthetic amphiphatic peptide, synthetic cationic peptide, synthetic polycationic peptide, synthetic hydrophobic peptide, or naturally occurring antimicrobial peptide, like sushi peptide and defensin. Moreover, the present invention relates to nucleic acid molecules encoding said fusion protein, vectors comprising said nucleic acid molecules and host cells comprising either said nucleic acid molecules or said vectors. In addition, the present invention relates to said fusion protein for use as a medicament, diagnostic means, cosmetic substance, a disinfectant or a food additive. The present invention also relates to the treatment or prevention of Gram-positive and/or Gram-negative bacterial contamination of foodstuff, of food processing equipment, of food processing plants, of surfaces coming into contact with foodstuff, of medical devices, of surfaces in hospitals and surgeries.

The present invention relates to a fusion protein composed of an enzyme having the activity of degrading the cell wall of Gram-negative bacteria and/or Gram-positive bacteria and at least two peptide stretches fused to the enzyme at the N- or C-terminus, wherein the peptide stretches are distinct and selected from the group of synthetic amphiphatic peptide, synthetic cationic peptide, synthetic polycationic peptide, synthetic hydrophobic peptide, or naturally occurring antimicrobial peptide, like sushi peptide and defensin. Moreover, the present invention relates to nucleic acid molecules encoding said fusion protein, vectors comprising said nucleic acid molecules and host cells comprising either said nucleic acid molecules or said vectors. In addition, the present invention relates to said fusion protein for use as a medicament, diagnostic means, cosmetic substance, a disinfectant or a food additive. The present invention also relates to the treatment or prevention of Gram-positive and/or Gram-negative bacterial contamination of foodstuff, of food processing equipment, of food processing plants, of surfaces coming into contact with foodstuff, of medical devices, of surfaces in hospitals and surgeries.

Gram-negative bacteria possess an outer membrane, with its characteristic asymmetric bilayer as a hallmark. The outer membrane bilayer consists of an inner monolayer containing phospholipids (primarily phosphatidyl ethanolamine) and an outer monolayer that is mainly composed of a single glycolipid, lipopolysaccharide (LPS). There is an immense diversity of LPS structures in the bacterial kingdom and the LPS structure may be modified in response to prevailing environmental conditions. The stability of the LPS layer and interaction between different LPS molecules is mainly achieved by the electrostatic interaction of divalent ions (Mg²⁺, Ca²⁺) with the anionic components of the LPS molecule (phosphate groups in the lipid A and the inner core and carboxyl groups of KDO). Furthermore, the dense and ordered packing of the hydrophobic moiety of lipid A, favored by the absence of unsaturated fatty acids, forms a rigid structure with high viscosity. This makes it less permeable for lipophilic molecules and confers additional stability to the outer membrane (OM).

In contrast to Gram-negative bacteria, Gram-positive bacteria do not possess an outer membrane. The cytoplasmic membrane is surrounded by an up to 25 nm thick layer of peptidoglycan (which is only up to 5 nm for Gram-negative bacteria) which forms the cell wall. Main purpose of the cell wall of Gram-positives is to maintain bacterial shape and to counteract the internal bacterial cell pressure. Peptidoglycan or murein is a polymer consisting of sugars and amino acids. The sugar component consists of alternating residues of β-(1,4) linked N-acetylglucosamine and N-acetylmuramic acid residues compose the sugar components. A peptide chain of three to five amino acids is attached to the N-acetylmuramic acid. The peptide chain can be cross-linked to the peptide chain of another strand forming a 3D mesh-like layer. The peptide chain may contain D- and L-amino acid residues and the composition may vary for different bacteria.

Various types of agents having bactericidal or bacteriostatic activity are known, e.g. antibiotics, endolysins, antimicrobial peptides and defensins. Increasingly microbial resistance to antibiotics, however, is creating difficulties in treating more and more infections caused by bacteria. Particular difficulties arise with infections caused by Gram-negative bacteria like Pseudomonas aeruginosa and Enterobacteriaceae and with infections caused by Gram-positive bacteria like Staphylococcus aureus, Enterococci, Streptococci, Listeria monocytogenes and Clostridium difficile, especially with e.g. Methicillin-resistant Staphylococcus aureus and Vancomycin-resistant Enterococci.

Endolysins are peptidoglycan hydrolases encoded by bacteriophages (or bacterial viruses). They are synthesized during late gene expression in the lytic cycle of phage multiplication and mediate the release of progeny virions from infected bacterial cells through degradation of the bacterial peptidoglycan. They are either β(1,4)-glycosylases, transglycosylases, amidases or endopeptidases. Antimicrobial application of endolysins was already suggested in 1991 by Gasson (GB2243611). Although the killing capacity of endolysins has been known for a long time, the use of these enzymes as antibacterials was ignored due to the success and dominance of antibiotics. Only after the appearance of multiple antibiotic resistant bacteria this concept of combating human pathogens with endolysins received interest. A compelling need to develop totally new classes of antibacterial agents emerged and endolysins used as ‘enzybiotics’—a hybrid term of ‘enzymes’ and ‘antibiotics’—seem to meet this need. In 2001, Fischetti and coworkers demonstrated for the first time the therapeutic potential of bacteriophage Cl endolysin towards group A streptococci (Nelson et al., 2001). Since then many publications have established endolysins as an attractive and complementary alternative to control bacterial infections, particularly by Gram-positive bacteria. Subsequently different endolysins against other Gram-positive pathogens such as Streptococcus pneumoniae (Loeffler et al., 2001), Bacillus anthracia (Schuch et al., 2002), S. agalactiae (Cheng et al., 2005) and Staphylococcus aureus (Rashel et al, 2007) have proven their efficacy as enzybiotics. Nowadays, the most important challenge of endolysin therapy lies in the insensitivity of Gram-negative bacteria towards the exogenous action of endolysins, since the outer membrane shields the access of endolysins from the peptidoglycan. This currently prevents the expansion of the range of effective endolysins to important Gram-negative pathogens. However, it is also known that endolysins can, under some conditions (e.g. high ionic strength), create stable protoplast, where the internal bacterial cell pressure is not sufficient to lead to a cell burst. Under these conditions the bacterial cell wall can regenerate and the bacteria will survive. Up to now endolysins are thus not qualified to substitute antibiotics.

Antimicrobial peptides (AMPs) represent a wide range of short, cationic or amphiphatic, gene encoded peptide antimicrobials that can be found in virtually every organism. Different AMPs display different properties, and many peptides in this class are being intensively researched not only as antimicrobials, but also as templates for cell penetrating peptides. Despite sharing a few common features (e.g. cationicity, amphiphaticity and short size), AMP sequences vary greatly, and at least four structural groups (α-helical, (β-sheet, extended and looped) have been proposed to accommodate the diversity of the observed AMP conformations. Likewise, several modes of action as antimicrobials have been proposed, and it was shown e.g. that the primary target of many of these peptides is the cell membrane whereas for other peptides the primary target is cytoplasmic invasion and disruption of core metabolic functions. AMPs may become concentrated enough to exhibit cooperative activity despite the absence of specific target binding; for example, by forming a pore in the membrane, as is the case for most AMPs. However, this phenomenon has only been observed in model phospholipid bilayers, and in some cases, AMP concentrations in the membrane that were as high as one peptide molecule per six phospholipid molecules were required for these events to occur. These concentrations are close to, if not at, full membrane saturation. As the minimum inhibitory concentration (MIC) for AMPs are typically in the low micromolar range, scepticism has understandably arisen regarding the relevance of these thresholds and their importance in vivo (Melo et al., Nature reviews, Microbiology, 2009, 245).

Defensins are a large family of small, cationic or amphiphatic, cysteine- and arginine-rich antimicrobial peptides, found in both vertebrates and invertebrates. Defensins are divided into five groups according to the spacing pattern of cysteines: plant, invertebrate, α-, β-, and θ-defensins. The latter three are mostly found in mammals. α-defensins are proteins found in neutrophils and intestinal epithelia. β-defensins are the most widely distributed and are secreted by leukocytes and epithelial cells of many kinds. θ-defensins have been rarely found so far e.g. in leukocytes of rhesus macaques. Defensins are active against bacteria, fungi and many enveloped and nonenveloped viruses. However, the concentrations needed for efficient killing of bacteria are mostly high, i.e. in the μ-molar range. Activity of many peptides may be limited in presence of physiological salt conditions, divalent cations and serum. Depending on the content of hydrophobic amino acid residues defensins also show haemolytic activity.

Thus, there is a need for new antimicrobial agents.

This object is solved by the subject matter defined in the claims.

The following figures serve to illustrate the invention.

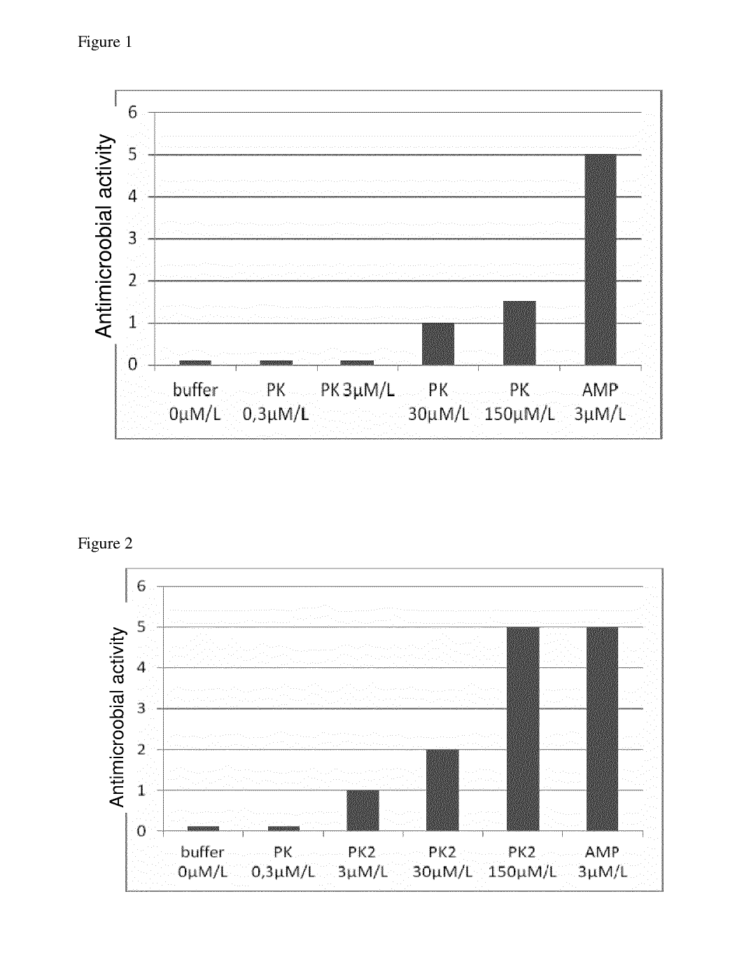

FIG. 1 shows a bar chart representing the antibacterial activities of a polycationic peptide named PK (SEQ ID NO: 23) at different concentrations compared to buffer (10 mM HEPES, 0.5 mM EDTA; pH 7.4) and an antimicrobial peptide (AMP=SMAP-29; SEQ ID NO: 45). The antibacterial activities are given as relative inactivation in logarithmic units on the y-axis.

FIG. 2 shows a bar chart representing the antibacterial activities shows the antibacterial activities of a polycationic peptide named PK2 (SEQ ID NO: 34) at different concentrations compared to buffer (10 mM HEPES, 0.5 mM EDTA; pH 7.4) and an antimicrobial peptide (AMP=SMAP-29, SEQ ID NO: 45). The antibacterial activities are given as relative inactivation in logarithmic units on the y-axis.

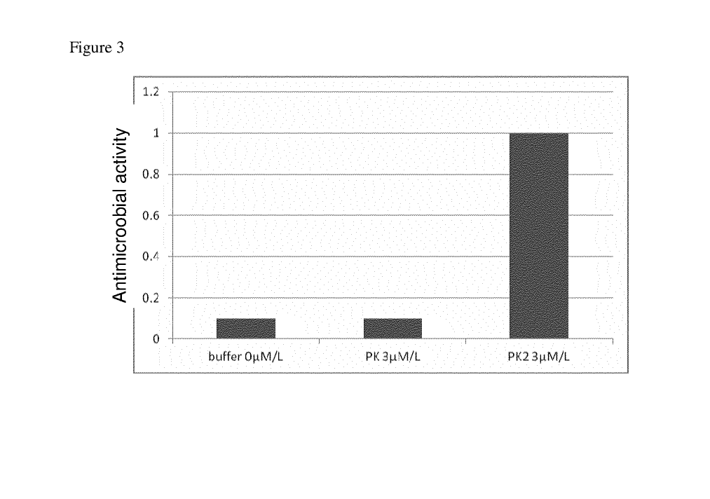

FIG. 3 shows a bar chart representing the antibacterial activities of a polycationic peptide named PK (SEQ ID NO: 23) and a polycationic peptide named PK2 (SEQ ID NO: 34) at a concentration of 3 μM/L compared to buffer (10 mM HEPES, 0.5 mM EDTA; pH 7.4). The antibacterial activities are given as relative inactivation in logarithmic units on the y-axis.

The term “protein” as used herein refers to a linear polymer of amino acid residues linked by peptide bonds in a specific sequence. The amino acid residues of a protein may be modified by e.g. covalent attachments of various groups such as carbohydrates and phosphate. Other substances may be more loosely associated with the protein, such as heme or lipid, giving rise to the conjugated proteins which are also comprised by the term “protein” as used herein. The various ways in which the protein fold have been elucidated, in particular with regard to the presence of alpha helices and beta-pleated sheets. The term “protein” as used herein refers to all four classes of proteins being all-alpha, all-beta, alpha/beta and alpha plus beta. Moreover, the term “protein” refers to a complex, wherein the complex refers to a homomer.

The term “fusion protein” as used herein refers to an expression product resulting from the fusion of three nucleic acid sequences. Such a protein may be produced, e.g. in recombinant DNA expression systems. Moreover, the term “fusion protein” as used herein refers to a fusion of a first amino acid sequence e.g. an endolysin, with a second and a third amino acid sequence. The second and third amino acid sequences are preferably peptide stretches, in particular selected from the group consisting of cationic, polycationic, hydrophobic, amphiphatic, sushi and antimicrobial peptides. Preferably, said second and third amino acid sequence is foreign to and not substantially homologous with any domain of the first amino acid sequence. Moreover, the fusion protein of the present invention also refers to an expression product resulting from the fusion of at least three nucleic acid sequences. The fusion protein also refers to a fusion of a first amino acid sequence e.g. an endolysin, with more than two additional amino acid sequences encoding peptide stretches. The amino acid sequences encoding peptide stretches of the fusion protein may be distinct from each other or the same.

The term “peptide stretch” as used herein refers to any kind of peptide linked to a protein such as an endolysin. In particular the term “peptide stretch” as used herein refers to a cationic peptide, a polycationic peptide, an amphiphatic peptide, a hydrophobic peptide, a sushi peptide and/or an antimicrobial peptide. However, a peptide stretch in the meaning of the present invention does not refer to tags like His-tags, preferably Hisₛ-tags, His₆-tags, His₇-tags, His_{g}-tags, His₉-tags, His₁₀-tags, His₁₁-tags, His₁₂-tags, His₁₆-tags and His₂₀-tags, Strep-tags, Avi-tags, Myc-tags, Gst-tags, JS-tags, cystein-tags, FLAG-tags or other tags known in the art, thioredoxin or maltose binding proteins (MBP). The term “tag” in contrast to the term “peptide stretch” as used herein refers to a peptide which can be useful to facilitate expression and/or affinity purification of a polypeptide, to immobilize a polypeptide to a surface or to serve as a marker or a label moiety for detection of a polypeptide e.g. by antibody binding in different ELISA assay formats as long as the function making the tag useful for one of the above listed facilitation is not caused by the positively charge of said peptide. However, the His₆-tag may, depending on the respective pH, also be positively charged, but is used as affinity purification tool as it binds to immobilized divalent cations and is not used as a peptide stretch according to the present invention.

The term “first peptide stretch” as used herein refers to a peptide stretch which is linked to the enzyme on the N- or C-terminus of said enzyme.

The term “second peptide stretch” as used herein refers to a peptide stretch which is linked to the first peptide stretch on the N- or C-terminus of said first peptide stretch.

The term “peptide” as used herein refers to short polypeptides consisting of from about 2 to about 100 amino acid residues, more preferably from about 4 to about 50 amino acid residues, more preferably from about 5 to about 30 amino acid residues, wherein the amino group of one amino acid residue is linked to the carboxyl group of another amino acid residue by a peptide bond. A peptide may have a specific function. A peptide can be a naturally occurring peptide or a synthetically designed and produced peptide. The peptide can be, for example, derived or removed from a native protein by enzymatic or chemical cleavage, or can be prepared using conventional peptide synthesis techniques (e.g. solid phase synthesis) or molecular biology techniques (see Sambrook, J. et al., Molecular Cloning: A Laboratory Manual, Cold Spring Harbor Press, Cold Spring Harbor, N.Y. (1989)). Preferred naturally occurring peptides are e.g. antimicrobial peptides, defensins, and sushi peptides. Preferred synthetically produced peptides are e.g. polycationic, amphipathic or hydrophobic peptides. A peptide in the meaning of the present invention does not refer to His-tags, Strep-tags, thioredoxin or maltose binding proteins (MBP) or the like, which are used to purify or locate proteins.

The term “endolysin” as used herein refers to an enzyme which has a peptidoglycan degrading effect and is naturally encoded by bacteriophages or bacterial viruses and which is suitable to degrade bacterial cell walls. “Endolysins” comprise at least one “enzymatically active domain” (EAD) having at least one of the following activities: endopeptidase, N-acetyl-muramoyl-L-alanine-amidase (amidase), N-acetyl-muramidase, N-acetyl-glucosaminidase or transglycosylases. In addition, the endolysins may contain also regions which are enzymatically inactive and bind to the cell wall of the host bacteria, wherein said regions are CBDs (cell wall binding domains). The endolysin may contain two or more CBDs. Generally, the cell wall binding domain is able to bind different components on the surface of bacteria. Preferably, the cell wall binding domain is a peptidoglycan binding domain and binds to the bacteria's peptidoglycan structure. The different domains of an endolysin can be connected by a domain linker.

The term “domain linker” as used herein refers to an amino acid sequence functioning to connect single protein domains with one another. As a rule domain linkers form no or only few regular secondary structures like α-helices or β-sheets and can occupy different conformations with the respective structural context. Methods to detect domain linker and properties of linker sequences are well known in the art as e.g. described in Bae et al., 2005, Bioinformatics, 21, 2264-2270 or George & Heringa, 2003, Protein Engineering, 15, 871-879.

The term “deletion” as used herein refers to the removal of 1, 2, 3, 4, 5 or more amino acid residues from the respective starting sequence.

The term “insertion” or “addition” as used herein refers to the insertion or addition of 1, 2, 3, 4, 5 or more amino acid residues to the respective starting sequence.

The term “substitution” as used herein refers to the exchange of an amino acid residue located at a certain position for a different one.

The term, cell wall” as used herein refers to all components that form the outer cell enclosure of the Gram-positive and Gram-negative bacteria and thus guarantee their integrity. In particular, the term, cell wall” as used herein refers to peptidoglycan, the outer membrane of the Gram-negative bacteria with the lipopolysaccharide, the bacterial cell membrane, but also to additional layers deposited on the peptidoglycan as e.g. capsules, outer protein layers or slimes.

The term “EAD” as used herein refers to the enzymatically active domain of an endolysin. The EAD is responsible for hydrolysing bacterial peptidoglycans. It exhibits at least one enzymatic activity of an endolysin. The EAD can also be composed of more than one enzymatically active module. The term “EAD” is used herein synonymously with the term “catalytic domain”.

As used herein, the term “cationic peptide” refers to a synthetic peptide having positively charged amino acid residues. Preferably a cationic peptide has a pKa-value of 9.0 or greater. Typically, at least four of the amino acid residues of the cationic peptide can be positively charged, for example, lysine or arginine. “Positively charged” refers to the side chains of the amino acid residues which have a net positive charge at about physiological conditions. The term “cationic peptide” as used herein refers also to polycationic peptides.

The term “polycationic peptide” as used herein refers to a synthetically produced peptide composed of mostly positively charged amino acid residues, in particular lysine and/or arginine residues. A peptide is composed of mostly positively charged amino acid residues of at least about 20, 30, 40, 50, 60, 70, 75, 80, 85, 90, 95 or about 100% of the amino acid residues are positively charged amino acid residues, in particular lysine and/or arginine residues. The amino acid residues being not positively charged amino acid residues can be neutrally charged amino acid residues and/or negatively charged amino acid residues and/or hydrophobic amino acid residues. Preferably the amino acid residues being not positively charged amino acid residues are neutrally charged amino acid residues, in particular serine and/or glycine.

The term, “antimicrobial peptide” (AMP) as used herein refers to any naturally occurring peptide that has microbicidal and/or microbistatic activity on for example bacteria, viruses, fungi, yeasts, mycoplasma and protozoa. Thus, the term “antimicrobial peptide” as used herein refers in particular to any peptide having anti-bacterial, anti-fungal, anti-mycotic, anti-parasitic, anti-protozoal, anti-viral, anti-infectious, anti-infective and/or germicidal, algicidal, amoebicidal, microbicidal, bactericidal, fungicidal, parasiticidal, protozoacidal, protozoicidal properties. The antimicrobial peptide may be a member of the RNAse A super family, a defensin, cathelicidin, granulysin, histatin, psoriasin, dermicidine or hepcidin. The antimicrobial peptide may be naturally occurring in insects, fish, plants, arachnids, vertebrates or mammals. Preferably the antimicrobial peptide may be naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human.

The term “sushi peptide” as used herein refers to complement control proteins (CCP) having short consensus repeats. The sushi module of sushi peptides functions as a protein-protein interaction domain in many different proteins. Peptides containing a Sushi domain have been shown to have antimicrobial activities. Preferably, sushi peptides are naturally occurring peptides.

The term “amphiphatic peptide” as used herein refers to synthetic peptides having both hydrophilic and hydrophobic functional groups. Preferably, the term “amphiphatic peptide” as used herein refers to a peptide having a defined arrangement of hydrophilic and hydrophobic groups e.g. amphiphatic peptides may be e.g. alpha helical, having predominantly non polar side chains along one side of the helix and polar residues along the remainder of its surface.

The term “hydrophobic group” as used herein refers to chemical groups such as amino acid side chains which are substantially water insoluble, but soluble in an oil phase, with the solubility in the oil phase being higher than that in water or in an aqueous phase. In water, amino acid residues having a hydrophobic side chain interact with one another to generate a nonaqueous environment. Examples of amino acid residues with hydrophobic side chains are valine, isoleucine, leucine, methionine, phenylalanine, tryptophan, cysteine, alanine, tyrosine, histidine, threonine, serine, proline and glycine residues.

The term “autolysins” refers to enzymes related to endolysins but encoded by bacteria and involved in e.g. cell division. An overview of autolysins can be found in “Bacterial peptidoglycan (murein) hydrolases. Vollmer W, Joris B, Charlier P, Foster S. FEMS Microbiol Rev. 2008 March; 32(2):259-86”.

The term “bacteriocin” as used herein refers to protein-like, polypeptide-like or peptide-like substances which are able to inhibit the growth of other bacteria. Some bacteriocins are capable of degrading bacterial cell walls like Lysostaphin (degrading Staphylococcus cell walls), Mutanolysin (degrading Streptococcus cell walls) and Enterolysin (degrading Enterococcus cell walls). Preferably said inhibition is specifically by means of absorption of said other bacteria to specific receptors of the bacteriocin. In general, bacteriocins are produced by microorganisms. However, the term “bacteriocin” as used herein refers both to an isolated form procuded by a microorganism or to a synthetically produced form, and refers also to variants which substantially retain the activities of their parent bacteriocins, but whose sequences have been altered by insertion or deletion of one or more amino acid residues.

The present invention relates to new antibacterial agents against Gram-positive and/or Gram-negative bacteria, in particular to fusion proteins composed of an enzyme having the activity of degrading the cell wall of Gram-positive and/or Gram-negative bacteria and two or more peptide stretches. These peptide stretches may be the same or distinct. These peptide stretches are linked to the N- or C-terminus of the enzyme of the fusion protein according to the present invention. The skilled person understands that the fusion proteins according to the present invention comprises 2, 3, 4, 5 or more peptide stretches.

In a preferred embodiment the fusion proteins according to the present invention are composed of an enzyme having the activity of degrading the cell wall of Gram-positive and/or Gram-negative bacteria and two peptide stretches fused to the enzyme on the N- or C-terminus. Preferably, wherein the peptide stretches are distinct from each other or the same.

The inventors found out that the fusion proteins according to the present invention show different spectra in view of the species to be effected.

In one aspect of the present invention the enzyme having the activity of degrading the cell wall of Gram-positive and/or Gram-negative bacteria is an endolysin, autolysin or bacteriocin. The enzyme of the fusion protein according to the present invention is no lysozyme. In a preferred embodiment the enzyme of the fusion protein according to the present invention is encoded by bacteriophages or bacterial virus, in particular an endolysin.

Preferred fusion proteins according to the present invention are depicted in SEQ ID NO: 70 to 77 and 127 to 147 may comprise one or more additional amino acid residues on the N-terminus. Preferably the additional amino acid residue is methionine.

Preferably, the endolysin is encoded by bacteriophages specific for Gram-negative bacteria such as Gram-negative bacteria of bacterial groups, families, genera or species comprising strains pathogenic for humans or animals like Enterobacteriaceae (Escherichia, especially E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (Pseudomonas, especially P. aeruginosa, Burkholderia, Stenotrophomonas, Shewanella, Sphingomonas, Comamonas), Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (Treponema and Borrelia), Leptospiraceae, Campylobacter, Helicobacter, Spirillum, Streptobacillus, Bacteroidaceae (Bacteroides, Fusobacterium, Prevotella, Porphyromonas), Acinetobacter, especially A. baumanii.

Preferably, the autolysin is encoded by Gram-negative bacteria such as Gram-negative bacteria of bacterial groups, families, genera or species comprising strains pathogenic for humans or animals as listed above.

The bacteriocin is preferably specific for Gram-negative bacteria as listed above, but may also be less specific.

The enzyme according to the present invention has cell wall degrading activity against Gram-negative bacteria of bacterial groups, families, genera or species comprising strains pathogenic for humans or animals as listed above.

In another preferred embodiment, the endolysin is encoded by bacteriophages specific for Gram-positive bacteria such as Gram-positive bacteria of bacterial groups, families, genera or species comprising strains pathogenic for humans or animals as listed in the following table.

TABLE 1
I. Phylum Actinobacteria

Class: Actinobacteridae

Order Actinomycetales

Families:
Actinomycineae: Actinomycetaceae (Actinomyces,
Mobiluncus)
Corynebacterineae: Mycobacteriaceae (Mycobacterium),
Nocardiaceae, Corynebacteriaceae
Frankineae: Frankiaceae
Micrococcineae: Brevibacteriaceae
Propionibacteriaceae (Propionibacterium)
Order: Bifidobacteriales
Families:
Bifidobacteriaceae (Bifidobacterium, Falcivibrio,
Gardnerella)
Other subclasses:Acidimicrobidae, Coriobacteridae,

Rubrobacteridae, Sphaerobacteridae

II. Phylum Firmicutes

Class: Bacilli

Order: Bacillales:

Families:
Bacillaceae (Bacillus), Listeriaceae (Listeria),
Staphylococcaceae
(Staphylococcus, Gemella, Jeotgalicoccus)

Order: Lactobacillales:

Families: Enterococcaceae (Enterococcus), Lactobacillaceae
(Lactobacillus, Pediococcus), Leuconostocaceae
(Leuconostoc),
Streptococcaceae (Lactococcus, Streptococcus)

Class: Clostridia

Order: Clostridiales (Clostridium, Peptostreptococcus,
Selenomonas)
Order: Halanaerobiales
Order: Thermoanaerobacterales

Class: Tenericutes/Mollicutes

Order: Mycoplasmatales (Mycoplasma, Ureaplasma)
Order: Entomoplasmatales (Spiroplasma)
Order: Anaeroplasmatales (Erysipelothrix)
Order: Acholeplasmatales (Acholeplasma)

Order: Haloplasmatales (Haloplasma)

In another preferred embodiment, the autolysin or bacteriocin is encoded by Gram-positive bacteria such as Gram-positive bacteria of bacterial groups, families, genera or species comprising strains pathogenic for humans or animals as listed in table 1.

In a preferred embodiment, the enzyme according to the present invention has cell wall degrading activity against Gram-positive bacteria of bacterial groups, families, genera or species comprising strains pathogenic for humans or animals like Listeria monocytogenes, Staphylococcus aureus, Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracia, Bacillus cereus, Propionibacterium acnes, Mycobacterium avium, Mycobacterium tuberculosis, Corynebacterium diphteriae, Mycoplasma pneumoniae, Actinomyces.

Examples for the endolysin part are listed in the following table:

TABLE 2

| | | Wild type | |
|---|---|---|---|
| phage | publication | endolysin | predicted function of the endolysin |
| φV10 | Perry, L.L. and Applegate, B.M. | PhiV10p30 | chitinase |
| FELS-1 | McClelland, M. and Wilson, R.K. | STM0907.Fels0 | chitinase |
| ε15 | Kropinksi, A.M. and McConnel, | epsilon15p25 | chitinase |
| | M.R. | | |
| YUA | Ceyssens. P. (Laboratory for | YuA20 | lytic transglycosylase (C)/1 transmembranair |
| | Gene technology) | | domain (N) |
| B3 | Braid, M.D. and Kitts, C.L. | ORF23 | lytic transglycosylase (C)/2 transmembranair |
| | | | domains (N) |
| BCEPμ | Summer, E.J. and Young, R. | BcepMu22 | lytic transglycosylase (M)/1 transmembranair |
| | | | domain (N) |
| F116 | Byrne, M. and Kropinski, A.M. | F116p62 | muraminidase (T4-like) |
| FELS-2 | McClelland, M. and Wilson, R.K. | STM2715.S.Fels2 | muraminidase (T4-like) |
| ES18 | Casjens, S.R. and Hendrix, R.W. | gp76 | muraminidase (T4-like) |
| SETP3 | De Lappe, N and Cormican, M. | SPSV3_gp23 | muraminidase (T4-like) |
| φECO32 | Savalia, D and Severinov, K | phi32_17 | muraminidase (T4-like) |
| HK022 | Juhala, R and Hendrix, R.W. | HK022p54 | muraminidase (lambdalike) |
| HK97 | Juhala, R and Hendrix, R.W. | HK97p58 | muraminidase (lambdalike) |
| HK620 | Clark, A.J. and Dhillon, T.S. | HK620p36 | muraminidase (lambdalike) |
| E1 | Pickard, D. and Dougan, G | VIP0007 | muraminidase (lambdalike) |
| SF6 | Casjens, S and Clark, A.J. | Sf6p62 | muraminidase (lambdalike) |
| SFV | Allison, G.E. and Verma, N.K. | R (SfVp40) | muraminidase (lambdalike) |
| BCEPC6B | Summer, EJ and Young, R. | gp22 | muraminidase (lambdalike) |
| BCEPNAZGUL | Summer, EJ and Young, R. | Nazgul38 | muraminidase (lambdalike) |
| P2 | Christie, G.E. and Calender, R. | K (P2p09) | muraminidase (lambdalike) |
| Wφ | Christie, G.E. and Esposito, D. | K (Wphi09) | muraminidase (lambdalike) |
| RV5 | Kropinski, A.M. and Johnson | rv5_gp085 | muraminidase (lambdalike) |
| JS98 | Zuber, S and Denou, E. | EpJS98_gp116 | muraminidase (T4-like) |
| 13A | Savalia, D and Molineux, I. | gp3.5 | muramoyl-L-alanine amidase |
| BA14 | Savalia, D and Molineux, I. | gp3.5 | muramoyl-L-alanine amidase |
| ECODS1 | Savalia, D and Molineux, I. | gp3.5 | muramoyl-L-alanine amidase |
| K1F | Scholl, D and Merril, C | CKV1F_gp16 | muramoyl-L-alanine amidase |
| T3 | Pajunen, M.I. and Mollineux, I.J. | T3p18 | muramoyl-L-alanine amidase |
| GH-1 | Kropinski, A.M. and Kovalyova, | gh-1p12 | muramoyl-L-alanine amidase |
| | I.V. | | |
| K11 | Molineux, I. and Savalia, D. | gp3.5 | muramoyl-L-alanine amidase |
| φCTX | Nakayama, K and Hayashi, T. | ORF12 | PG-binding domain (N)/muramidase (C) |
| BCEP43 | Summer, EJ and Young, R. | Bcep43-27 | PG-binding domain (N)/muramidase (C) |
| BCEP781 | Summer, EJ and Young, R. | Bcep781-27 | PG-binding domain (N)/muramidase (C) |
| BCEP1 | Summer, EJ and Young, R. | Bcep1-28 | PG-binding domain (N)/muramidase (C) |
| BCEPNY3 | Summer, EJ and Young, R. | BcepNY3gene26 | PG-binding domain (N)/muramidase (C) |
| φE12-2 | DeShazer, D and Nierman, W.C. | gp45 | PG-binding domain (N)/muramidase (C) |
| φ52237 | DeShazer, D and Nierman, W.C. | gp28 | PG-binding domain (N)/muramidase (C) |
| φP27 | Recktenwald, J and Schmidt, H. | P27p30 | endopeptidase |
| RB49 | Monod, C and Krisch, H.M. | RB49p102 | endopeptidase |
| φ1 | Arbiol, C. and Comeau, A.M. | phi1-p102 | endopeptidase |
| T5 | Pankova, N.V. and Ksenzenko, | lys (T5.040) | endopeptidase |
| | V.N. | | |
| 201phi2-1 | Thomas et al., 2008 | | PG-binding domain (N)/unknown catalytic |
| | | | domain (C) |
| Aeh1 | Monod, C and Krisch, H.M. | Aeh1p339 | muraminidase (T4-like) |
| YYZ-2008 | Kropinski, A.M. | YYZgp45 | muraminidase (lambda-like) |

Also preferred is the endolysin part deriving from endolysins of the Pseudomonas aeruginosa phages DKZ and EL, of the Pseudomonas putida phage, of the E. coli phage N4, of the phage LUZ24, gp61 muramidase, STM0016 endolysin, PSP3 endolysin and endolysin of Salmonella enteritis phage PVPSE1.

Further preferred endolysins are Listeria phage endolysins PlyA118, PlyA500, PlyPSA, PlyA511, PlyP35, PlyP40, Staphylococcal phage Phi 11 endolysin, Phi MR11 endolysin, LysK, Clostridium perfringens PlyS6, Ply3626, Clostridium difficile: CD27L endolysin, Streptococcus: B30 endolysin, phage Dp-1 Pal amidase, C1 endolysin, Cpl-1 endolysin, PlyGBS, Enterococccus: PlyV12, Bacillus anthracia: Phage gamma endolysin PlyG.

Preferred autolysins are described in: Bacterial peptidoglycan (murein) hydrolases. Vollmer W, Joris B, Charlier P, Foster S. FEMS Microbiol Rev. 2008 March; 32(2):259-86. Epub 2008 Feb 11. Review. An example of a preferred autolysin is the At1A Autolysine.

Preferred bacteriocins are Lysostaphin (degrading Staphylococcus cell walls), Mutanolysin (degrading Streptococcus cell walls) and Enterolysin (degrading Enterococcus cell walls).

Further examples for the endolysin part is selected from the group consisting of Cpl-1 according to SEQ ID NO: 1, Ply511 according to SEQ ID NO: 2, LysK according to SEQ ID NO: 3, Lysostaphin according to SEQ ID NO: 4, PA6-gp20 according to SEQ ID NO: 5, phiKZgp144 according to SEQ ID NO: 6, ELgp188 according to SEQ ID NO: 7, Salmonella endolysin according to SEQ ID NO:8, Enterobacteria phage T4 endolysin according to SEQ ID NO: 9, Acinetobacter baumanii endolysin according to SEQ ID NO: 10, E. coli Phage K1F endolysin according to SEQ ID NO: 11, OBPgpLYS according to SEQ ID NO: 12, PSP3 Salmonella endolysin (PSP3gp10) according to SEQ ID NO:13, E. coli Phage P2 endolysin (P2gp09) according to SEQ ID NO: 14, Salmonella typhimurium phage muramidase STM0016 according to SEQ ID NO:15, E. coli Phage N4 muramidase N4-gp61 according to SEQ ID NO: 16 and N4-gp61 trunc. according to SEQ ID NO: 17, KZ144 according to SEQ ID NO: 18.

In another preferred embodiment of the present invention the endolysins, autolysins and bacteriocins of the fusion protein according to the present invention comprise modifications and/or alterations of the amino acid sequences. Such alterations and/or modifications may comprise mutations such as deletions, insertions and additions, substitutions or combinations thereof and/or chemical changes of the amino acid residues, e.g. biotinylation, acetylation, pegylation, chemical changes of the amino-, SH- or carboxyl-groups. Said endolysins, autolysins and bacteriocins of the fusion protein according to the present invention exhibit the lytic activity of the respective wild-type endolysin, autolysin and bacteriocin. However, said activity can be the same, higher or lower as the activity of the respective wild-type endolysin, autolysin and bacteriocin. Said activity can be about 10, 20, 30, 40, 50, 60, 70, 80, 90, 100, 110, 120, 130, 140, 150, 160, 170, 180, 190 or about 200% of the activity of the respective wild-type endolysin, autolysin and bacteriocin or even more. The activity can be measured by assays well known in the art by a person skilled in the art as e.g. the plate lysis assay or the liquid lysis assay which are e.g. described in Briers et al., J. Biochem. Biophys Methods 70: 531-533, (2007) or Donovan D M, Lardeo M, Foster-Frey J. FEMS Microbiol Lett. 2006 December; 265(1) or similar publications.

The peptide stretches of the fusion protein according to the present invention may be linked to the enzyme by additional amino acid residues e.g. due to cloning reasons. The peptide stretches of the fusion protein according to the present invention may be linked to each other by additional amino acid residues. Preferably, said additional amino acid residues may be not recognized and/or cleaved by proteases. Preferably said peptide stretches may be linked to each other and/or to the enzyme by at least 1, 2, 3, 4, 5, 6, 7, 8, 9 or 10 additional amino acid residues. In a preferred embodiment the second peptide stretch is linked to the first peptide stretch which is fused to the N- or C-terminus of the enzyme by the additional amino acid residues glycine, serine and serine (Gly-Ser-Ser), glycine, alanine, glycine and alanine (Gly-Ala-Gly-Ala), glycine, alanine, glycine, alanine, glycine, alanine, glycine and alanine (Gly-Ala-Gly-Ala-Gly-Ala-Gly-Ala) or glycine, alanine, glycine, alanine, glycine, alanine, glycine, alanine, glycine, alanine, glycine and alanine (Gly-Ala-Gly-Ala-Gly-Ala-Gly-Ala-Gly-Ala-Gly-Ala). Moreover, the second peptide stretch fused on the N-terminus of the first peptide stretch of the fusion protein according to the invention further comprises additional amino acids on its N-terminus. Preferably the peptide stretch comprises the amino acid methionine (Met), or methionine, glycine and serine (Met-Gly-Ser). In another preferred embodiment the first peptide stretch is linked to the N-terminus of the enzyme by additional amino acid residues, in particular glycine and serine (Gly-Ser) and the second peptide stretch is linked to the N-terminus of the first peptide stretch by additional amino acid residues, in particular glycine and serine (Gly-Ser) or glycine, serine and serine (Gly-Ser-Ser). In another preferred embodiment the second peptide stretch is linked to the N- or C-terminus of the first peptide stretch by additional amino acid residues glycine, serine and serine (Gly-Ser-Ser), glycine, alanine, glycine and alanine (Gly-Ala-Gly-Ala), glycine, alanine, glycine, alanine, glycine, alanine, glycine and alanine (Gly-Ala-Gly-Ala-Gly-Ala-Gly-Ala) or glycine, alanine, glycine, alanine, glycine, alanine, glycine, alanine, glycine, alanine, glycine and alanine (Gly-Ala-Gly-Ala-Gly-Ala-Gly-Ala-Gly-Ala-Gly-Ala). In another preferred embodiment the first peptide stretch is linked to the C-terminus of the enzyme by additional amino acid residues, in particular glycine and serine (Gly-Ser) and the second peptide stretch is linked to the C-terminus of the first peptide stretch by additional amino acid residues, in particular glycine and serine (Gly-Ser).

The peptide stretches of the fusion protein according to the present invention are preferably covalently bound to the enzyme. Preferably, said peptide stretches consist of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acid residues. Especially preferred are peptide stretches comprising about 5 to about 100 amino acid residues, about 5 to about 50 or about 5 to about 30 amino acid residues. More preferred are peptide stretches comprising about 6 to about 42 amino acid residues, about 6 to about 39 amino acid residues, about 6 to about 38 amino acid residues, about 6 to about 31 amino acid residues, about 6 to about 25 amino acid residues, about 6 to about 24 amino acid residues, about 6 to about 22 amino acid residues, about 6 to about 21 amino acid residues, about 6 to about 20 amino acid residues, about 6 to about 19 amino acid residues, about 6 to about 16 amino acid residues, about 6 to about 14 amino acid residues, about 6 to about 12 amino acid residues, about 6 to about 10 amino acid residues or about 6 to about 9 amino acid residues.

Preferably, the peptide stretches are no tag such as a His-tag, Strep-tag, Avi-tag, Myc-tag, Gst-tag, JS-tag, cystein-tag, FLAG-tag or other tags known in the art and no thioredoxin or maltose binding proteins (MBP). However, the fusion protein according to the present invention may comprise in addition such tag or tags.

More preferably the peptide stretches have the function to lead the fusion protein through the outer membrane but may have activity or may have no or only low activity when administered without being fused to the enzyme. The function to lead the fusion protein through the outer membrane of Gram-negative bacteria is caused by the potential of the outer membrane or LPS disrupting or permeabilising or destabilizing activity of said peptide stretches. Such outer membrane or LPS disrupting or permeabilising or destabilizing activity of the peptide stretches may be determined in a method as follows: The bacteria cells to be treated are cultured in liquid medium or on agar plates. Then the bacteria cell concentration in the liquid medium is determined photometrically at OD600 nm or the colonies on the agar plates are counted, respectively. Now, the bacteria cells in liquid medium or on the plates are treated with a fusion protein according to the invention. After incubation the bacteria cell concentration in the liquid medium is determined photometrically at OD600 nm or the colonies on the agar plates are counted again. If the fusion protein exhibits such outer membrane or LPS disrupting or permeabilising or destabilizing activity, the bacteria cells are lysed due to the treatment with the fusion protein and thus, the bacteria cell concentration in the liquid medium or the number of the bacteria colonies on the agar plate is reduced. Thus, the reduction in bacteria cell concentration or in the number of bacteria colonies after treatment with fusion protein is indicative for an outer membrane or LPS disrupting or permeabilising or destabilizing activity of the fusion protein.

In a preferred embodiment the peptide stretches of the fusion protein according to the present invention are selected from the group of cationic peptides, polycationic peptides, hydrophobic peptides, antimicrobial peptides, sushi peptides and amphiphatic peptides. The two or more peptide stretches at the N- or C-terminus of the fusion protein according to the invention may be distinct cationic peptides or polycationic peptides or antimicrobial peptides or amphiphatic peptides or hydrophobic peptides. The two or more peptide stretches at the N- or C-terminus of the fusion protein according to the invention may be any combination of a cationic peptide, a polycationic peptide, a hydrophobic peptide, an antimicrobial peptide, a sushi peptide and an amphiphatic peptide.

Especially preferred are cationic and/or polycationic peptide stretches comprising at least one motive according to SEQ ID NO: 19 (KRKKRK). In particular cationic peptide stretches comprising at least 2, 3, 4, 5, 6, 7, 8, 9, 10, 11, 12, 13, 14, 15, 16 or 17 motives according to SEQ ID NO: 19 (KRKKRK) are preferred. More preferred are cationic peptide stretches comprising at least one KRK motive (lys-arg-lys), preferable at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 KRK motives.

In another preferred embodiment of the present invention the cationic peptide stretch comprises beside the positively charged amino acid residues, in particular lysine and/or arginine residues, neutrally charged amino acid residues, in particular glycine and/or serine residues. Preferred are cationic peptide stretches consisting of about 70% to about 100%, or about 80% to about 95%, or about 85% to about 90% positively charged amino acid residues, in particular lysine, arginine and/or histidine residues, more preferably lysine and/or arginine residues and of about 0% to about 30%, or about 5% to about 20%, or about 10% to about 20% neutrally charged amino acid residues, in particular glycine and/or serine residues. Preferred are polypeptide stretches consisting of about 4% to about 8% serine residues, of about 33% to about 36% arginine residues and of about 56% to about 63% lysine residues. Especially preferred are polypeptide stretches comprising at least one motive according to SEQ ID NO: 20 (KRXKR), wherein X is any other amino acid than lysine, arginine and histidine. Especially preferred are polypeptide stretches comprising at least one motive according to SEQ ID NO: 21 (KRSKR). More preferred are cationic stretches comprising at least about 2, 3, 4, 5, 6, 7, 8, 9, 10, 11, 12, 13, 14, 15, 16, 17, 18, 19 or about 20 motives according to SEQ ID NO: 20 (KRXKR) or SEQ ID NO: 21 (KRSKR).

Also preferred are polypeptide stretches consisting of about 9 to about 16% glycine residues, of about 4 to about 11% serine residues, of about 26 to about 32% arginine residues and of about 47 to about 55% lysine residues. Especially preferred are polypeptide stretches comprising at least one motive according to SEQ ID NO: 22 (KRGSG). More preferred are cationic stretches comprising at least about 2, 3, 4, 5, 6, 7, 8, 9, 10, 11, 12, 13, 14, 15, 16, 17, 18, 19 or about 20 motives according to SEQ ID NO: 22 (KRGSG).

In another preferred embodiment of the present invention the cationic peptide stretch comprises beside the positively charged amino acid residues, in particular lysine and/or arginine residues, hydrophobic amino acid residues, in particular valine, isoleucine, leucine, methionine, phenylalanine, tryptophan, cysteine, alanine, tyrosine, histidine, threonine, serine, proline and glycine residues, more preferably alanine, valine, leucine, isoleucine, phenylalanine, and/or tryptophan residues. Preferred are cationic peptide stretches consisting of about 70% to about 100%, or about 80% to about 95%, or about 85% to about 90% positively charged amino acid residues, in particular lysine and/or arginine residues and of about 0% to about 30%, or about 5% to about 20%, or about 10% to about 20% hydrophobic amino acid residues, valine, isoleucine, leucine, methionine, phenylalanine, tryptophan, cysteine, alanine, tyrosine, histidine, threonine, serine, proline and glycine residues, more preferably alanine, valine, leucine, isoleucine, phenylalanine, and/or tryptophan residues.

Examples for cationic and polycationic peptide stretches are listed in the following table:

TABLE 3

| peptide stretch | length | SEQ ID NO: |
|---|---|---|
| KRKKRK | 6 | SEQ ID NO: 19 |
| KRKKRKKRK | 9 | SEQ ID NO: 23 |
| RRRRRRRRR | 9 | SEQ ID NO: 24 |
| KKKKKKKK | 8 | SEQ ID NO: 25 |
| KRKKRKKRKK | 10 | SEQ ID NO: 26 |
| KRKKRKKRKKRK | 12 | SEQ ID NO: 27 |
| KRKKRKKRKKRKKR | 14 | SEQ ID NO: 28 |
| KKKKKKKKKKKKKKKK | 16 | SEQ ID NO: 29 |
| KRKKRKKRKKRKKRKKRK | 18 | SEQ ID NO: 30 |
| KRKKRKKRKKRKKRKKRKK | 19 | SEQ ID NO: 31 |
| RRRRRRRRRRRRRRRRRRR | 19 | SEQ ID NO: 32 |
| KKKKKKKKKKKKKKKKKKK | 19 | SEQ ID NO: 33 |
| KRKKRKKRKRSKRKKRKKRK | 20 | SEQ ID NO: 34 |
| KRKKRKKRKRSKRKKRKKRKK | 21 | SEQ ID NO: 35 |
| KRKKRKKRKKRKKRKKRKKRK | 21 | SEQ ID NO: 36 |
| KRKKRKKRKRGSGKRKKRKKRK | 22 | SEQ ID NO: 37 |
| KRKKRKKRKRGSGSGKRKKRKKRK | 24 | SEQ ID NO: 38 |
| KRKKRKKRKKRKKRKKRKKRKKRKK | 25 | SEQ ID NO: 39 |
| KRKKRKKRKRSKRKKRKKRKRSKRKKRKKRK | 31 | SEQ ID NO: 40 |
| KRKKRKKRKRGSGSGKRKKRKKRKGSGSGKRKKRKKRK | 38 | SEQ ID NO: 41 |
| KRKKRKKRKKRKKRKKRKKRKKRKKRKKRKKRKKRKKRK | 39 | SEQ ID NO: 42 |
| KRKKRKKRKRSKRKKRKKRKRSKRKKRKKRKRSKRKKRKKRK | 42 | SEQ ID NO: 43 |

In a further aspect of the present invention at least one of the fused peptide stretches is an antimicrobial peptide, which comprises a positive net charge and around 50% hydrophobic amino acids. The antimicrobial peptides are amphiphatic with a length of about 12 to about 50 amino acid residues. The antimicrobial peptides are naturally occurring in insects, fish, plants, arachnids, vertebrates or mammals. Preferably the antimicrobial peptide may be naturally occurring in radish, silk moth, wolf spider, frog, preferably in Xenopus laevis, Rana frogs, more preferably in Rana catesbeiana, toad, preferably Asian toad Bufo bufo gargarizans, fly, preferably in Drosophila, more preferably in Drosophila melanogaster, in Aedes aegypti, in honey bee, bumblebee, preferably in Bombus pascuorum, flesh fly, preferably in Sarcophaga peregrine, scorpion, horseshoe crab, catfish, preferably in Parasilurus asotus, cow, pig, sheep, porcine, bovine, monkey and human.

In another preferred embodiment of the present invention the antimicrobial peptide stretches consisting of about 0% to about 5%, or about 0% to about 35%, or about 10% to about 35% or about 15% to about 45%, or about 20% to about 45% positively charged amino acid residues, in particular lysine and/or arginine residues and of about 50% to about 80%, or about 60% to about 80%, or about 55% to about 75%, or about 70% to about 90% hydrophobic amino acid residues, valine, isoleucine, leucine, methionine, phenylalanine, tryptophan, cysteine, alanine, tyrosine, histidine, threonine, serine, proline and glycine residues, more preferably alanine, valine, leucine, isoleucine, phenylalanine, and/or tryptophan residues.

In another preferred embodiment of the present invention the antimicrobial peptide stretches consisting of about 4% to about 58% positively charged amino acid residues, in particular lysine and/or arginine residues and of about 33% to about 89% hydrophobic amino acid residues, valine, isoleucine, leucine, methionine, phenylalanine, tryptophan, cysteine, alanine, tyrosine, histidine, threonine, serine, proline and glycine residues, more preferably alanine, valine, leucine, isoleucine, phenylalanine, and/or tryptophan residues.

Examples for antimicrobial peptides according to the present invention are listed in the following table.

TABLE 4

| | | SEQ ID |
|---|---|---|
| Peptide | Sequence | NO |
| LL-37 | LLGDFFRKSKEKIGKEFKRI | SEQ ID |
| | VQRIKDFLRNLVPRTES | NO: 44 |
| SMAP-29 | RGLRRLGRKIAHGVKKYGPT | SEQ ID |
| | VLRIIRIAG | NO: 45 |
| Indolicidin | ILPWKWPWWPWRR | SEQ ID |
| | | NO: 46 |
| Protegrin | RGGRLCYCRRRFCVCVGR | SEQ ID |
| | | NO: 47 |
| Cecropin P1 | SWLSKTAKKLENSAKKRISE | SEQ ID |
| | GIAIAIQGGPR | NO: 48 |
| Magainin | GIGKFLHSAKKFGKAFVGEI | SEQ ID |
| | MNS | NO: 49 |
| Pleurocidin | GWGSFFKKAAHVGKHVGKAA | SEQ ID |
| | LTHYL | NO: 50 |
| Cecropin A | GGLKKLGKKLEGAGKRVFNA | SEQ ID |
| (A. aegypti) | AEKALPVVAGAKALRK | NO: 51 |
| Cecropin A (D. | GWLKKIGKKIERVGQHTRDA | SEQ ID |
| melanogaster) | TIQGLGIPQQAANVAATARG | NO: 52 |
| Buforin II | TRSSRAGLQFPVGRVHRLLR | SEQ ID |
| | K | NO: 53 |
| Sarcotoxin IA | GWLKKIGKKIERVGQHTRDA | SEQ ID |
| | TIQGLGIAQQAANVAATAR | NO: 54 |
| Apidaecin | ANRPVYIPPPRPPHPRL | SEQ ID |
| | | NO: 55 |
| Ascaphine 5 | GIKDWIKGAAKKLIKTVASH | SEQ ID |
| | IANQ | NO: 56 |
| Nigrocine 2 | GLLSKVLGVGKKVLCGVSGL | SEQ ID |
| | VC | NO: 57 |
| Pseudin 1 | GLNTLKKVFQGLHEAIKLIN | SEQ ID |
| | NHVQ | NO: 58 |
| Ranalexin | FLGGLIVPAMICAVTKKC | SEQ ID |
| | | NO: 59 |
| Melittin | GIGAVLKVLTTGLPALISWI | SEQ ID |
| | KRKRQQ | NO: 60 |
| Lycotoxin 1 | IWLTALKFLGKHAAKKLAKQ | SEQ ID |
| | QLSKL | NO: 61 |
| Parasin 1 | KGRGKQGGKVRAKAKTRSS | SEQ ID |
| | | NO: 62 |
| Buforin I | AGRGKQGGKVRAKAKTRSSR | SEQ ID |
| | AGLQFPVGRVHRLLRKGNY | NO: 78 |
| Dermaseptin 1 | ALWKTMLKKLGTMALHAGKA | SEQ ID |
| | ALGAAADTISQGTQ | NO: 79 |
| Bactenecin 1 | RLCRIVVIRVCR | SEQ ID |
| | | NO: 80 |
| Thanatin | GSKKPVPIIYCNRRTGKCQR | SEQ ID |
| | M | NO: 81 |
| Brevinin 1T | VNPIILGVLPKVCLITKKC | SEQ ID |
| | | NO: 82 |
| Ranateurin 1 | SMLSVLKNLGKVGLGFVACK | SEQ ID |
| | INIKQC | NO: 83 |
| Esculentin 1 | GIFSKLGRKKIKNLLISGLK | SEQ ID |
| | NVGKEVGMDVVRTGIKIAGC | NO: 84 |
| | KIKGEC | |
| Tachyplesin | RWCFRVCYRGICYRKCR | SEQ ID |
| | | NO: 85 |
| Androctonin | RSVCRQIKICRRRGGCYYKC | SEQ ID |
| | TNRPY | NO: 86 |
| alpha-defensin | DCYCRIPACIAGERRYGTCI | SEQ ID |
| | YQGRLWAFCC | NO: 87 |
| beta-defensin | NPVSCVRNKGICVPIRCPGS | SEQ ID |
| | MKQIGTCVGRAVKCCRKK | NO: 88 |
| theta-defensin | GFCRCLCRRGVCRCICTR | SEQ ID |
| | | NO: 89 |
| defensin | ATCDLLSGTGINHSACAAHC | SEQ ID |
| (sapecin A) | LLRGNRGGYCNGKAVCVCRN | NO: 90 |
| Thionin | TTCCPSIVARSNFNVCRIPG | SEQ ID |
| (crambin) | TPEAICATYTGCIIIPGATC | NO: 91 |
| | PGDYAN | |
| defensin from | QKLCQRPSGTWSGVCGNNNA | SEQ ID |
| radish | CKNQCIRLEKARHGSCNYVF | NO: 92 |
| | PAHCICYFPC | |
| Drosomycin | DCLSGRYKGPCAVWDNETCR | SEQ ID |
| | RVCKEEGRSSGHCSPSLKCW | NO: 93 |
| | CEGC | |
| Hepcidin | DTHFPICIFCCGCCHRSKCG | SEQ ID |
| | MCCKT | NO: 94 |
| Bac 5 | RFRPPIRRPPIRPPFYPPFR | SEQ ID |
| | PPIRPPIFPPIRPPFRPPLG | NO: 95 |
| | RPFP | |
| PR-39 | RRRPRPPYLPRPRPPPFFPP | SEQ ID |
| | RLPPRIPPGFPPRFPPRFP | NO: 96 |
| Pyrrhocoricin | VDKGSYLPRPTPPRPIYNRN | SEQ ID |
| | | NO: 97 |
| Histatin 5 | DSHAKRHHGYKRKFHEKHHS | SEQ ID |
| | HRGY | NO: 98 |

In a further aspect of the present invention at least one of the fused peptide stretches is a sushi peptide which is described by Ding J L, Li P, Ho B Cell Mol Life Sci. 2008 April; 65(7-8):1202-19. The Sushi peptides: structural characterization and mode of action against Gram-negative bacteria. Especially preferred is the sushi 1 peptide according to SEQ ID NO: 63.

Preferred sushi peptides are sushi peptides S1 and S3 and multiples thereof; FASEB J. 2000 September; 14(12):1801-13.

In a further aspect of the present invention at least one of the fused peptide stretches is a hydrophobic peptide, which comprises at least 90% of the hydrophobic amino acid residues of valine, isoleucine, leucine, methionine, phenylalanine, tryptophan, cysteine, alanine, tyrosine, histidine, threonine, serine, proline and/or glycine. In another preferred embodiment the hydrophobic peptide fused to the fusion protein of the invention consist of about 90% to about 95%, or of about 90% to about 100%, or of about 95% to about 100% of the hydrophobic amino acid residues of valine, isoleucine, leucine, methionine, phenylalanine, tryptophan, cysteine, alanine, tyrosine, histidine, threonine, serine, proline and/or glycine.

Preferred hydrophobic peptides are Walmagh1 having the amino acid sequence according to SEQ ID NO: 65 and the hydrophobic peptide having the amino acid sequence Phe-Phe-Val-Ala-Pro (SEQ ID NO: 66).

In a further aspect of the present invention at least one of the fused peptide stretches is an amphiphatic peptide, which comprises one or more of the positively charged amino acid residues of lysine, arginine and/or histidine, combined to one or more of the hydrophobic amino acid residues of valine, isoleucine, leucine, methionine, phenylalanine, tryptophan, cysteine, alanine, tyrosine, histidine, threonine, serine, proline and/or glycine. Side chains of the amino acid residues are oriented in order that cationic and hydrophobic surfaces are clustered at opposite sides of the peptide. Preferably, more than about 30, 40, 50, 60 or 70% of the amino acids in said peptide are positively charged amino acids. Preferably, more than about 30, 40, 50, 60 or 70%, of the amino acid residues in said peptide are hydrophobic amino acid residues. Advantageously, the amphiphatic peptide is fused at the N-terminal or the C-terminal end of the enzyme having cell wall degrading activity, thus enhancing the amphiphaticity of the latter proteins.

In another embodiment of the invention, the amphiphatic peptide consists of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acid residues. In a preferred embodiment at least about 30, 40, 50, 60 or 70% of the said amino acid residues of the amphiphatic peptide are either arginine or lysine residues and/or at least about 30, 40, 50, 60 or 70% of the said amino acid residues of the amphiphatic peptide are of the hydrophobic amino acids valine, isoleucine, leucine, methionine, phenylalanine, tryptophan, cysteine, alanine, tyrosine, histidine, threonine, serine, proline and/or glycine.

In another preferred embodiment of the present invention the amphiphatic peptide stretch comprises beside the positively charged amino acid residues, in particular lysine and/or arginine residues, hydrophobic amino acid residues, in particular valine, isoleucine, leucine, methionine, phenylalanine, tryptophan, cysteine, alanine, tyrosine, histidine, threonine, serine, proline and glycine residues, more preferably alanine, valine, leucine, isoleucine, phenylalanine, and/or tryptophan residues. Preferred are amphiphatic peptide stretches consisting of about 10% to about 50%, or about 20% to about 50%, or about 30% to about 45% or about 5% to about 30% positively charged amino acid residues, in particular lysine and/or arginine residues and of about 50% to about 85%, or about 50% to about 90%, or about 55% to about 90%, or about 60% to about 90%, or about 65% to about 90% hydrophobic amino acid residues, valine, isoleucine, leucine, methionine, phenylalanine, tryptophan, cysteine, alanine, tyrosine, histidine, threonine, serine, proline and glycine residues, more preferably alanine, valine, leucine, isoleucine, phenylalanine, and/or tryptophan residues. In another preferred embodiment amphiphatic peptide stretches consisting of 12% to about 50% positively charged amino acid residues, in particular lysine and/or arginine residues and of about 50% to about 85% hydrophobic amino acid residues, valine, isoleucine, leucine, methionine, phenylalanine, tryptophan, cysteine, alanine, tyrosine, histidine, threonine, serine, proline and glycine residues, more preferably alanine, valine, leucine, isoleucine, phenylalanine, and/or tryptophan residues.

Preferred amphiphatic peptides are α4-helix of T4 lysozyme according to SEQ ID NO: 68 and WLBU2-Variant having the amino acid sequence according to SEQ ID NO: 64 and Walmagh 2 according to SEQ ID NO: 69.

In another preferred embodiment of the present invention the peptide stretches of the fusion protein according to the present invention comprise modifications and/or alterations of the amino acid sequences. Such alterations and/or modifications may comprise mutations such as deletions, insertions and additions, substitutions or combinations thereof and/or chemical changes of the amino acid residues, e.g. biotinylation, acetylation, pegylation, chemical changes of the amino-, SH- or carboxyl-groups.

In a preferred embodiment the two peptide stretches of the fusion protein on the N- or C-terminus according to the present invention consist of a polycationic peptide combined with an amphiphatic peptide. The amphiphatic peptide may be the α4-helix of T4-lysozyme according to SEQ ID NO: 68, WLBU2-Variant according to SEQ ID NO: 64 or Walmagh 2 according to SEQ ID NO: 69. In a preferred embodiment the polycationic peptide may be a peptide according to SEQ ID NO: 23 or a peptide according to SEQ ID NO: 34. In another preferred embodiment the amphiphatic peptide may be α4-helix of T4-lysozyme according to SEQ ID NO: 68 and the polycationic peptide may be a peptide according to SEQ ID NO: 23. In another preferred embodiment the amphiphatic peptide may be Walmagh 2 according to SEQ ID NO: 69 and the polycationic peptide may be a peptide according to SEQ ID NO: 23. In another preferred embodiment the amphiphatic peptide may be WLBU2-Variant according to SEQ ID NO: 64 and the polycationic peptide may be a peptide according to SEQ ID NO: 23. The amphiphatic and polycationic peptide of the fusion protein according to the present invention are arranged successively at the N- or C-terminus. The order of the amphiphatic and polycationic peptide on the N- or C-terminus does not matter, e.g. starting at the N-terminus of the fusion protein the amphiphatic peptide is followed by the polycationic peptide which is followed by the enzyme, or the polycationic peptide is followed by the amphiphatic peptide which is followed by the enzyme. Alternatively, the enzyme is followed by polycationic peptide which is followed by the amphiphatic peptide or the enzyme is followed by the amphiphatic peptide which is followed by the polycationic peptide, so that both peptides are arranged at the C-terminus of the enzyme.

In another preferred embodiment the amphiphatic peptide α4-helix of T4-lysozyme according to SEQ ID NO: 68 may be combined with the polycationic peptide according to SEQ ID NO: 23. In another preferred embodiment the amphiphatic peptide Walmagh 2 according to SEQ ID NO: 69 may be combined with the polycationic peptide according to SEQ ID NO: 23. In another preferred embodiment the amphiphatic peptide WLBU2-Variant according to SEQ ID NO: 64 may be combined with the polycationic peptide according to SEQ ID NO: 23.

In a preferred embodiment the two peptide stretches of the fusion protein on the N- or C-terminus according to the present invention consist of a polycationic peptide combined with a hydrophobic peptide. In a preferred embodiment the hydrophobic peptide may be the pentapeptide according to SEQ ID NO: 66 or Walmagh 1 according to SEQ ID NO: 65. In a preferred embodiment the polycationic peptide may be a peptide according to SEQ ID NO: 23 or a peptide according to SEQ ID NO: 34. In another preferred embodiment the hydrophobic peptide may be the pentapeptide according to SEQ ID NO: 66 and the polycationic peptide may be a peptide according to SEQ ID NO: 23. In another preferred embodiment the hydrophobic peptide may be Walmagh 1 according to SEQ ID NO: 65 and the polycationic peptide may be a peptide according to SEQ ID NO: 23. The hydrophobic and polycationic peptide of the fusion protein according to the present invention are arranged successively at the N- or C-terminus. The order of the hydrophobic and polycationic peptide on the N- or C-terminus does not matter, e.g. starting at the N-terminus of the fusion protein the hydrophobic peptide is followed by the polycationic peptide which is followed by the enzyme, or the polycationic peptide is followed by the hydrophobic peptide which is followed by the enzyme. Alternatively, the enzyme is followed by polycationic peptide which is followed by the hydrophobic peptide or the enzyme is followed by hydrophobic peptide which is followed by the polycationic peptide, so that both peptides are arranged at the C-terminus of the enzyme.

In another preferred embodiment the hydrophobic peptide pentapeptide according to SEQ ID NO: 66 may be combined with the polycationic peptide according to SEQ ID NO: 23. In another preferred embodiment the hydrophobic peptide Walmagh 1 according to SEQ ID NO: 65 may be combined with the polycationic peptide according to SEQ ID NO: 23.

In another preferred embodiment the two peptide stretches of the fusion protein on the N- or C-terminus according to the present invention consist of a polycationic peptide combined with an antimicrobial peptide. Preferably, the antimicrobial peptide may be Parasin 1 according to SEQ ID NO: 62, Lycotoxin 1 according to SEQ ID NO: 61, SMAP-29 according to SEQ ID NO: 45, LL37 according to SEQ ID NO: 44, Melittin according to SEQ ID NO: 60, Sarcotoxin IA according to SEQ ID NO: 54, Pseudin1 according to SEQ ID NO: 58, Indolicidin according to SEQ ID NO: 46, Buforin II according to SEQ ID NO: 53, Magainin according to SEQ ID NO: 49 or Cecropin A (A. aegyptii) according to SEQ ID NO: 51. In a preferred embodiment the polycationic peptide may be a peptide according to SEQ ID NO: 23 or a peptide according to SEQ ID NO: 34. In another preferred embodiment the antimicrobial peptide may be Parasin 1 according to SEQ ID NO: 62 and the polycationic peptide may be a peptide according to SEQ ID NO: 23. In another preferred embodiment the antimicrobial peptide may be Lycotoxin 1 according to SEQ ID NO: 61 and the polycationic peptide may be a peptide according to SEQ ID NO: 23. In another preferred embodiment the antimicrobial peptide may be SMAP-29 according to SEQ ID NO: 45 and the polycationic peptide may be a peptide according to SEQ ID NO: 23. In another preferred embodiment the antimicrobial peptide may be Cecropin A (A. aegyptii) according to SEQ ID NO: 51 and the polycationic peptide may be a peptide according to SEQ ID NO: 34. The antimicrobial and polycationic peptide of the fusion protein according to the present invention are arranged successively at the N- or C-terminus. The order of the antimicrobial and polycationic peptide on the N- or C-terminus does not matter, e.g. starting at the N-terminus of the fusion protein the antimicrobial peptide is followed by the polycationic peptide which is followed by the enzyme, or the polycationic peptide is followed by the antimicrobial peptide which is followed by the enzyme. Alternatively, the enzyme is followed by polycationic peptide which is followed by the antimicrobial peptide or the enzyme is followed by the antimicrobial peptide which is followed by the polycationic peptide, so that both peptides are arranged at the C-terminus of the enzyme.

In another preferred embodiment the antimicrobial peptide Parasin 1 according to SEQ ID NO: 62 may be combined with the polycationic peptide according to SEQ ID NO: 23. In another preferred embodiment the antimicrobial peptide Lycotoxin 1 according to SEQ ID NO: 61 may be combined with the polycationic peptide according to SEQ ID NO: 23. In another preferred embodiment the antimicrobial peptide SMAP-29 according to SEQ ID NO: 45 may be combined with the polycationic peptide according to SEQ ID NO: 23. In another preferred embodiment the antimicrobial peptide Cecropin A (A. aegyptii) according to SEQ ID NO: 51 may be combined with the polycationic peptide according to SEQ ID NO: 34.

In another preferred embodiment the two peptide stretches of the fusion protein on the N- or C-terminus according to the present invention consist of two distinct antimicrobial peptides. Preferably, the antimicrobial peptides may be Parasin 1 according to SEQ ID NO: 62, Lycotoxin 1 according to SEQ ID NO: 61, SMAP-29 according to SEQ ID NO: 45, LL37 according to SEQ ID NO: 44, Melittin according to SEQ ID NO: 60, Sarcotoxin IA according to SEQ ID NO: 54, Pseudin1 according to SEQ ID NO: 58, Indolicidin according to SEQ ID NO: 46, Buforin II according to SEQ ID NO: 53, Magainin according to SEQ ID NO: 49 or Cecropin A (A. aegyptii) according to SEQ ID NO: 51. In another preferred embodiment the antimicrobial peptide may be LL37 according to SEQ ID NO: 44 combined with the antimicrobial peptide SMAP-29 according to SEQ ID NO: 45. In another preferred embodiment the antimicrobial peptide may be SMAP-29 according to SEQ ID NO: 45 combined with the antimicrobial peptide Melittin according to SEQ ID NO: 60. In another preferred embodiment the antimicrobial peptide may be SMAP-29 according to SEQ ID NO: 45 combined with the antimicrobial peptide Sarcotoxin IA according to SEQ ID NO: 54. In another preferred embodiment the antimicrobial peptide may be Pseudin1 according to SEQ ID NO: 58 combined with the antimicrobial peptide Indolicidin according to SEQ ID NO: 46. In another preferred embodiment the antimicrobial peptide may be BuforinII according to SEQ ID NO: 53 combined with the antimicrobial peptide Maginin according to SEQ ID NO: 49. In another preferred embodiment the antimicrobial peptide may be Pseudin1 according to SEQ ID NO: 58 combined with the antimicrobial peptide LL37 according to SEQ ID NO: 44. The antimicrobial peptides of the fusion protein according to the present invention are arranged successively at the N- or C-terminus. The order of the antimicrobial peptides on the N- or C-terminus does not matter, e.g. starting at the N-terminus of the fusion protein the antimicrobial peptide is followed by the a different antimicrobial peptide which is followed by the enzyme, or the different antimicrobial peptide is followed by the antimicrobial peptide which is followed by the enzyme. Alternatively, the enzyme is followed by the antimicrobial peptide which is followed by a different antimicrobial peptide or the enzyme is followed by a different antimicrobial peptide which is followed by the antimicrobial peptide, so that both peptides are arranged at the C-terminus of the enzyme.

In a preferred embodiment of the present invention the fusion protein consists of two or more peptide stretches according to SEQ ID NO: 19 to 69 and 78 to 98 and an enzyme according to SEQ ID NO: 1 to 18. In a preferred embodiment of the present invention the fusion protein comprises two or more peptide stretches selected from the group of peptide stretches according to SEQ ID NO: 19 to 69 and 78 to 98 and an enzyme selected from the group of enzymes according to SEQ ID NO: 1 to 18. In another preferred embodiment of the present invention the fusion protein comprises two or more peptide stretches selected from the group of peptides stretches according to SEQ ID NO: 19 to 69 and 78 to 98 and an enzyme selected from the group of enzymes according to SEQ ID NO: 1 to 18, wherein the peptide stretches are fused to the N- or C-terminus of the enzyme.

Specific examples of fusion proteins according to the present invention are listed in the following table:

TABLE 3

| | Second peptide stretch | First peptide stretch (N-terminal unless | |
|---|---|---|---|
| Fusion protein | (N-terminal unless otherwise indicated) | otherwise indicated) | Enzyme part |
| SEQ ID NO: 70 | Pentapeptide (SEQ ID NO: 66) | polycationic peptide (SEQ ID NO: 23) | OBPgpLys (SEQ ID NO: 12) |
| SEQ ID NO: 76 | polycationic peptide (SEQ ID NO: 23) | Pentapeptide (SEQ ID NO: 66) | OBPgpLys (SEQ ID NO: 12) |
| SEQ ID NO: 71 | α4-helix of T4-lysozyme (SEQ ID NO: 68) | polycationic peptide (SEQ ID NO: 23) | OBPgpLys (SEQ ID NO: 12) |
| SEQ ID NO: 77 | polycationic peptide (SEQ ID NO: 23) | α 4-helix of T4-lysozyme(SEQ ID NO: 68) | OBPgpLys (SEQ ID NO: 12) |
| SEQ ID NO: 72 | Walmagh 1 (SEQ ID NO: 65) | polycationic peptide (SEQ ID NO: 23) | OBPgpLys (SEQ ID NO: 12) |
| SEQ ID NO: 73 | Walmagh 2 (SEQ ID NO: 69) | polycationic peptide (SEQ ID NO: 23) | OBPgpLys (SEQ ID NO: 12) |
| SEQ ID NO: 74 | Parasin 1 (SEQ ID NO: 62) | polycationic peptide (SEQ ID NO: 23) | OBPgpLys (SEQ ID NO: 12) |
| SEQ ID NO: 75 | Lycotoxin 1 (SEQ ID NO: 61) | polycationic peptide (SEQ ID NO: 23) | OBPgpLys (SEQ ID NO: 12) |
| SEQ ID NO: 127 | Polycationic peptide (SEQ ID NO: 23) | SMAP-29 (SEQ ID NO: 45) | KZ144 (SEQ ID NO: 18) |
| SEQ ID NO: 128 | SMAP-29 (SEQ ID NO: 45) | Polycationic peptide (SEQ ID NO: 23) | EL188 (SEQ ID NO: 7) |
| SEQ ID NO: 129 | SMAP-29 (SEQ ID NO: 45) | SMAP-29 (SEQ ID NO: 45) | KZ144 (SEQ ID NO: 18) |
| SEQ ID NO: 130 | SMAP-29 (SEQ ID NO: 45) | LL-37 (SEQ ID NO: 44) | N4gp61 (SEQ ID NO: 16) |
| SEQ ID NO: 131 | Sarcotoxin IA (SEQ ID NO: 54) | SMAP-29 (SEQ ID NO: 45) | Lysostaphin (SEQ ID NO: 4) |
| SEQ ID NO: 132 | (C-terminal of the enzyme) | (C-terminal of the second peptide-stretch) | Lysostaphin (SEQ ID NO: 4) |
| | Sarcotoxin IA (SEQ ID NO: 54) | SMAP-29 (SEQ ID NO: 45) | |
| SEQ ID NO: 133 | Melittin (SEQ ID NO: 60) | SMAP-29 (SEQ ID NO: 45) | N4gp61 (SEQ ID NO: 16) |
| SEQ ID NO: 134 | LL-37 (SEQ ID NO: 44) | Pseudin-1 (SEQ ID NO: 58) | Cpl-1 (SEQ ID NO: 1) |
| SEQ ID NO: 135 | Magainin (SEQ ID NO: 49) | Buforin II (SEQ ID NO: 53) | Ply511 (SEQ ID NO: 2) |
| SEQ ID NO: 136 | SMAP-29 (SEQ ID NO: 45) | LL37 (SEQ ID NO: 44) | STM0016 (SEQ ID NO: 15) |
| SEQ ID NO: 137 | Mellitin (SEQ ID NO: 60) | SMAP-29 (SEQ ID NO: 45) | STM0016 (SEQ ID NO: 15) |
| SEQ ID NO: 138 | Indolicidin (SEQ ID NO: 46) | Pseudin1 (SEQ ID NO: 58) | Cpl1 (SEQ ID NO: 1) |
| SEQ ID NO: 139 | (C-terminal of the second peptide stretch) | (C-terminal of the enzyme) | LysK (SEQ ID NO: 3) |
| | Cecropin A (A. aegyptii) (SEQ ID NO: 51) | PK2 (SEQ ID NO: 34) | |
| SEQ ID NO: 140 | (C-terminal of the second peptide stretch) | (C-terminal of the enzyme) | KZ144 (SEQ ID NO: 18) |
| | Polycationic peptide (SEQ ID NO: 23) | SMAP-29 (SEQ ID NO: 45) | |
| SEQ ID NO: 141 | (C-terminal of the second peptide stretch) | (C-terminal of the enzyme) | EL188 (SEQ ID NO: 7) |
| | SMAP-29 (SEQ ID NO: 45) | Polycationic peptide (SEQ ID NO: 23) | |
| SEQ ID NO: 142 | (C-terminal of the second peptide stretch) | (C-terminal of the enzyme) | STM0016 (SEQ ID NO: 15) |
| | SMAP-29 (SEQ ID NO: 45) | LL37 (SEQ ID NO: 44) | |
| SEQ ID NO: 143 | (C-terminal of the second peptide stretch) | (C-terminal of the enzyme) | STM0016 (SEQ ID NO: 15) |
| | Melittin (SEQ ID NO: 60) | SMAP-29 (SEQ ID NO: 45) | |
| SEQ ID NO: 144 | (C-terminal of the second peptide stretch) | (C-terminal of the enzyme) | Lysostaphin (SEQ ID NO: 4) |
| | Sarcotoxin IA (SEQ ID NO: 54) | SMAP-29 (SEQ ID NO: 45) | |
| SEQ ID NO: 145 | (C-terminal of the second peptide stretch) | (C-terminal of the enzyme) | Cpl1 (SEQ ID NO: 1) |
| | Indolicidin (SEQ ID NO: 46) | Pseudin1 (SEQ ID NO: 58) | |
| SEQ ID NO: 146 | (C-terminal of the second peptide stretch) | (C-terminal of the enzyme) | Ply511 (SEQ ID NO: 2) |
| | Magainin (SEQ ID NO: 49) | Buforinll (SEQ ID NO: 53) | |
| SEQ ID NO: 147 | PK2 (SEQ ID NO: 34) | Cecropin A (A. aegyptii) (SEQ ID NO: 51) | LysK (SEQ ID NO: 3) |

The fusion protein according to the present invention, and thus in particular the especially preferred fusion proteins according to SEQ ID NO: 70 to 77 and 127 to 147, may additional comprise a methionine on the N-terminus.

The fusion protein according to the present invention, and thus in particular the especially preferred fusion proteins according to SEQ ID NO: 70 to 77 and 127 to 147 may additional comprise a tag e.g. for purification. Preferred is a His₆-tag, preferably at the C-terminus and/or the N-terminus of the fusion protein. Said tag can be linked to the fusion protein by additional amino acid residues e.g. due to cloning reasons. Preferably said tag can be linked to the fusion protein by at least 1, 2, 3, 4, 5, 6, 7, 8, 9 or 10 additional amino acid residues. Preferably said additional amino acid residues may not be recognized and/or cleaved by proteases. In a preferred embodiment the fusion protein comprises a His₆-tag at its C-terminus linked to the fusion protein by the additional amino acid residues lysine and glycine (Lys-Gly) or leucine and glutamic acid (Leu-Glu). Preferably, said additional amino acid residues may be not recognized or cleaved by proteases. In another preferred embodiment the fusion protein comprises a His₆-tag at its N-terminus linked to the fusion protein by the additional amino acid residues lysine and glycine (Lys-Gly) or leucine and glutamic acid (Leu-Glu). In another preferred embodiment the fusion protein comprises a His₆-tag at its N- and C-terminus linked to the fusion protein by the additional amino acid residues lysine and glycine (Lys-Gly) or leucine and glutamic acid (Leu-Glu).

In a more preferred embodiment the fusion protein comprises a His₆-tag at its C-terminus linked to the fusion protein by the additional amino acid residues leucine and glutamic acid (Leu-Glu) and the second peptide stretch of the fusion protein according to the invention is linked to the N-terminus of the first peptide stretch by the additional amino acid residues glycine and serine (Gly-Ser), glycine, serine and serine (Gly-Ser-Ser), glycine, alanine, glycine and alanine (Gly-Ala-Gly-Ala), glycine, alanine, glycine, alanine, glycine, alanine, glycine and alanine (Gly-Ala-Gly-Ala-Gly-Ala-Gly-Ala) or glycine, alanine, glycine, alanine, glycine, alanine, glycine, alanine, glycine, alanine, glycine and alanine (Gly-Ala-Gly-Ala-Gly-Ala-Gly-Ala-Gly-Ala-Gly-Ala) and/or and the fusion protein comprises on the N-terminus the additional amino acid residues methionine (Met) or methionine and glycine (Met-Gly) or methionine, glycine and serine (Met-Gly-Ser). In another preferred embodiment the fusion protein comprises a His₆-tag at its C-terminus linked to the fusion protein by the additional amino acid residues leucine and glutamic acid (Leu-Glu) and the second peptide stretch of the fusion protein according to the invention is linked to the N-terminus of the first peptide stretch by the additional amino acid residues glycine and serine (Gly-Ser), glycine, serine and serine (Gly-Ser-Ser), glycine, alanine, glycine and alanine (Gly-Ala-Gly-Ala), glycine, alanine, glycine, alanine, glycine, alanine, glycine and alanine (Gly-Ala-Gly-Ala-Gly-Ala-Gly-Ala) or glycine, alanine, glycine, alanine, glycine, alanine, glycine, alanine, glycine, alanine, glycine and alanine (Gly-Ala-Gly-Ala-Gly-Ala-Gly-Ala-Gly-Ala-Gly-Ala) and the first peptide stretch is fused to the N-terminus of the enzyme by the additional amino acid residues glycine and serine (Gly-Ser), glycine, serine and serine (Gly-Ser-Ser), glycine, alanine, glycine and alanine (Gly-Ala-Gly-Ala), glycine, alanine, glycine, alanine, glycine, alanine, glycine and alanine (Gly-Ala-Gly-Ala-Gly-Ala-Gly-Ala) or glycine, alanine, glycine, alanine, glycine, alanine, glycine, alanine, glycine, alanine, glycine and alanine (Gly-Ala-Gly-Ala-Gly-Ala-Gly-Ala-Gly-Ala-Gly-Ala) and/or the fusion protein comprises on the N-terminus the additional amino acid residues methionine (Met) or methionine and glycine (Met-Gly) or methionine, glycine and serine (Met-Gly-Ser).

Fusion proteins are constructed by linking at least three nucleic acid sequences using standard cloning techniques as described e.g. by Sambrook et al. 2001, Molecular Cloning: A Laboratory Manual. Such a protein may be produced, e.g., in recombinant DNA expression systems. Such fusion proteins according to the present invention can be obtained by fusing the nucleic acids for endolysin and the respective peptide stretches.

The fusion proteins according to the present invention may be fused or linked to other additional proteins. Example for this other additional protein is thioredoxin.

The present invention further relates to an isolated nucleic acid molecule encoding the fusion protein according to the present invention. The present invention further relates to a vector comprising the nucleic acid molecule according to the present invention. Said vector may provide for the constitutive or inducible expression of said fusion protein according to the present invention.

The invention also relates to a method for obtaining said fusion proteins from a micro-organism, such as a genetically modified suitable host cell which expresses said fusion proteins. Said host cell may be a micro-organism such as bacteria or yeast or an animal cell as e.g. a mammalian cell, in particular a human cell. In one embodiment of the present invention the host cell is a Pichia pastoris cell. The host may be selected due to mere biotechnological reasons, e.g. yield, solubility, costs, etc. but may be also selected from a medical point of view, e.g. a non-pathological bacteria or yeast, human cells. One aspect of the present invention refers to a method for the production of a fusion protein comprising culturing a host cell comprising a nucleic acid sequence encoding a fusion protein according to the present invention under conditions supporting the expression of said fusion protein.

Another aspect of the present invention is related to a method for genetically transforming a suitable host cell in order to obtain the expression of the fusion proteins according to the invention wherein the host cell is genetically modified by the introduction of a genetic material encoding said fusion proteins into the host cell and obtain their translation and expression by genetic engineering methods well known by the man skilled in the art.

In a further aspect the present invention relates to a composition, preferably a pharmaceutical composition, comprising a fusion protein according to the present invention and/or a host transformed with a nucleic acid molecule or a vector comprising a nucleotide sequence encoding a fusion protein according to the present invention.

In a preferred embodiment of the present invention the composition comprises additionally agents permeabilizing the outer membrane of Gram-negative bacteria such metal chelators as e.g. EDTA, TRIS, lactic acid, lactoferrin, polymyxin, citric acid and/or other substances as described e.g. by Vaara (Agents that increase the permeability of the outer membrane. Vaara M. Microbiol. Rev. 1992 September; 56 (3):395-441). Also preferred are compositions comprising combinations of the above mentioned permeabilizing agents. Especially preferred is a composition comprising about 10 μM to about 100 mM EDTA, more preferably about 50 μM to about 10 mM EDTA, more preferably about 0.5 mM to about 10 mM EDTA, more preferably about 0.5 mM to about 2 mM EDTA, more preferably about 0.5 mM to 1 mM EDTA. However, also compositions comprising about 10 μM to about 0.5 mM EDTA are preferred. Also preferred is a composition comprising about 0.5 mM to about 2 mM EDTA, more preferably about 1 mM EDTA and additionally about 10 mM to about 100 mM TRIS. The present invention also relates to a fusion protein according to the present invention and/or a host transformed with a nucleic acid comprising a nucleotide sequence encoding a fusion protein according to the present invention for use as a medicament. In a further aspect the present invention relates to the use of a fusion protein according to the present invention and/or a host transformed with a vector comprising a nucleic acid molecule comprising a nucleotide sequence encoding a modified fusion protein according to the present invention in the manufacture of a medicament for the treatment and/or prevention of a disorder, disease or condition associated with Gram-positive and/or Gram-negative bacteria. In particular the treatment and/or prevention of the disorder, disease or condition may be caused by Gram-negative bacteria of bacterial groups, families, genera or species comprising strains pathogenic for humans or animals like Enterobacteriaceae (Escherichia, especially E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (Pseudomonas, especially P. aeruginosa, Burkholderia, Stenotrophomonas, Shewanella, Sphingomonas, Comamonas), Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (Treponema and Borrelia), Leptospiraceae, Campylobacter, Helicobacter, Spirillum, Streptobacillus, Bacteroidaceae (Bacteroides, Fusobacterium, Prevotella, Porphyromonas), Acinetobacter, especially A. baumanii. In particular the treatment and/or prevention of the disorder, disease or condition may be caused by Gram-positive bacteria of bacterial groups, families, genera or species comprising strains pathogenic for humans or animals like Listeria monocytogenes, Staphylococcus aureus, Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracia, Bacillus cereus, Propionibacterium acnes, Mycobacterium avium, Mycobacterium tuberculosis, Corynebacterium diphteriae, Mycoplasma pneumoniae, Actinomyces.

The present invention further relates to a medicament comprising a fusion protein according to the present invention and/or a host transformed with a nucleic acid comprising a nucleotide sequence encoding a fusion protein according to the present invention.

In a further aspect the present invention relates to a method of treating a disorder, disease or condition in a subject in need of treatment and/or prevention, which method comprises administering to said subject an effective amount of a fusion protein according to the present invention and/or an effective amount of a host transformed with a nucleic acid comprising a nucleotide sequence encoding a fusion protein according to the present invention or a composition according to the present invention. The subject may be a human or an animal.

In particular said method of treatment may be for the treatment and/or prevention of a disorder, disease or condition caused by Gram-positive and/or Gram-negative bacteria, in particular of bacterial infections caused by Gram-positive and/or Gram-negative bacteria.

In particular said method of treatment may be for the treatment and/or prevention of infections of the skin, of soft tissues, the respiratory system, the lung, the digestive tract, the eye, the ear, the teeth, the nasopharynx, the mouth, the bones, the vagina, of wounds of bacteraemia and/or endocarditis caused by Gram-positive and/or Gram-negative bacteria, in particular by the Gram-positive and/or Gram-negative bacteria as listed above.

The dosage and route of administration used in a method of treatment (or prophylaxis) according to the present invention depends on the specific disease/site of infection to be treated. The route of administration may be for example oral, topical, nasopharyngeal, parenteral, intravenous, rectal or any other route of administration.

For application of a fusion protein according to the present invention and/or an effective amount of a host transformed with a nucleic acid comprising a nucleotide sequence encoding a fusion protein according to the present invention or a composition according to the present invention to a site of infection or site endangered to be infected a formulation may be used that protects the active compounds from environmental influences such as proteases, oxidation, immune response etc., until it reaches the site of infection. Therefore, the formulation may be capsule, dragee, pill, powder, suppository, emulsion, suspension, gel, lotion, cream, salve, injectable solution, syrup, spray, inhalant or any other medical reasonable galenic formulation. Preferably, the galenic formulation may comprise suitable carriers, stabilizers, flavourings, buffers or other suitable reagents. For example, for topical application the formulation may be a lotion, cream, gel, salve or plaster, for nasopharyngeal application the formulation may be saline solution to be applied via a spray to the nose. For oral administration in case of the treatment and/or prevention of a specific infection site e.g. in the intestine, it can be necessary to protect a fusion protein according to the present invention from the harsh digestive environment of the gastrointestinal tract until the site of infection is reached. Thus, bacteria as carrier, which survive the initial steps of digestion in the stomach and which secret later on a fusion protein according to the present invention into the intestinal environment can be used.

In a specific embodiment of the present invention a fusion protein according to the present invention and/or a host transformed with a vector comprising a nucleic acid molecule comprising a nucleotide sequence encoding a fusion protein according to the present invention is used in the manufacture of a medicament for the treatment and/or prevention of a disorder, disease or condition caused by Pseudomonas, particularly by Pseudomonas aeruginosa in particular intestinal affections, in particular in infants, infections of the meninges, e.g. meningitis haemorrhagica, infections of the middle ear, the skin (Eethyma gangraenosum), in particular burns, the urinary tract, rhinitis, bacteremic pneumonia, in particular wherein the patient is suffering from cystic fibrosis or hematologic malignancies such as leukemia, or with neutropenia from immunosuppressive therapy, septicemia, in particular because of long-term intravenous or urinary catheterization, invasive surgical procedures and severe burns, endocarditis, in particular wherein the patient is a intravenous drug user or a patient with complications from open heart surgery, highly destructive ocular infections, in particular after the use of contaminated ophthalmologic solutions or severe facial burns, osteochondritis, in particular as a result of severe trauma or puncture wounds through contaminated clothing.

In another specific embodiment of the present invention the disorder, disease or condition is caused by Burkholderia pseudomallei, in particular Whitmore's Disease, chronic pneumonia, septicemia, in particular wherein the patient has a traumatized skin lesion.

In another specific embodiment of the present invention the disorder, disease or condition is caused by Salmonella thyphimurium and Salmonella enteritidis, in particular acute gastroenteritis and local purulent processes, particularly osteomyelitis, endocarditis, cholecystitis and especially caused by Salmonella thyphimurium meningitis, in particular wherein the patient is less than two years old.

In another specific embodiment of the present invention the disorder, disease or condition is caused by Salmonella typhi, in particular typus.

In another specific embodiment of the present invention the disorder, disease or condition is caused by Salmonell paratyphi, in particular paratyphus.

In another specific embodiment of the present invention the disorder, disease or condition is caused by Acinetobacter baumannii, in particular bronchitis, pneumonia, wound infections and septicemia, in particular as a result of intravenous catheterization.

In another specific embodiment of the present invention the disorder, disease or condition is caused by Escherichia coli, in particular extra intestinal infections, particularly appendicitis, purulent cholecystitis, peritonitis, purulent meningitis and infection of the urinary tract, intraintestinal E. coli infections, particularly epidemic enteritis, and infectious disease similar to dysentery, septicemia, enterotoxemia, mastitis and dysentery.

In another specific embodiment of the present invention the disorder, disease or condition is caused by Klebsiella pneumoniae, in particular pneumonia, bacteremia, meningitis and infections of the urinary tract.

In a specific embodiment of the present invention a fusion protein according to the present invention and/or a host transformed with a vector comprising a nucleic acid molecule comprising a nucleotide sequence encoding a fusion protein according to the present invention is used in the manufacture of a medicament for the treatment and/or prevention of a disorder, disease or condition caused by Listeria monocytogenes, in particular Granulomatosis infantiseptica (listeriosis of newborns), mononucleosis, conjunctivitis, meningitis, granulomatosis septica and the listeriosis of pregnant women.

In another specific embodiment of the present invention the disorder, disease or condition is caused by Staphylococcus aureus, in particular infections of the skin like pyoderma, particularly folliculitis, furuncle, carbuncle, abscesses of the sweat glands and pemphigus, and like scaled skin syndrome. The scaled skin syndrome can appear in three clinical pictures: dermatitis exfoliativa, impetigo bullosa and scarlatiniform erythroderma. Moreover the disorder, disease or condition caused by Staphylococcus aureus is Staphylococcus pneumonia, hospitalism, in particular surgical wound infections, mastitis puerperalis and enterokolitis, and food poisonings.

In another specific embodiment of the present invention the disorder, disease or condition is caused by Streptococcus pyogenes, in particular tonsillitis, pharyngitis, scarlet, erysipelas, rheumatic fever and acute glomerulonephritis.

In another specific embodiment of the present invention the disorder, disease or condition is caused by Streptococcus pneumoniae, in particular pneumonia, ulcus serpens corneae, otitis media, meningitis, peritonitis, mastoiditis and osteomyelitis.

In another specific embodiment of the present invention the disorder, disease or condition is caused by Clostridium perfringens, in particular gas gangrene, enteritis necroticans ulcerosa and food poisonings.

In another specific embodiment of the present invention the disorder, disease or condition is caused by Clostridium botulinum, in particular botulism.

In another specific embodiment of the present invention the disorder, disease or condition is caused by Clostridium difficile, in particular pseudomembranoes enterokolitis.

In another specific embodiment of the present invention the disorder, disease or condition is caused by Bacillus anthracia, in particular cutaneous anthrax, inhalation anthrax, and gastrointestinal anthrax.

In another specific embodiment of the present invention the disorder, disease or condition is caused by Enterococcus faecalis or Enterococcus faecium, like nosokomial infections and endokarditis.

In another specific embodiment of the present invention the disorder, disease or condition is caused by Bacillus cereus, in particular food poisonings, bronchial pneumonia, septicaemia and meningitis.

In another specific embodiment of the present invention the disorder, disease or condition is caused by Mycobacterium avium, Mycobacterium paratuberculosis and Mycobacterium tuberculosis, in particular tuberculosis.

In another specific embodiment of the present invention the disorder, disease or condition is caused by Mycoplasma pneumoniae, in particular pneumonia, diseases of the upper respiratory tract and inflammations of the ear drum.

In another specific embodiment of the present invention the disorder, disease or condition is caused by Actinomyces, in particular actinomycosis in human, cattle, cat and dog.

In another specific embodiment of the present invention the disorder, disease or condition is caused by Corynebacterium diphteriae, in particular localized diphtheria of the tonsils, the nose, the nasopharynx or the middle ear, progressive diphtheria of the larynx, the trachea and the bronchi, toxic or maligne diphtheria, skin and wound diphtheria.

Preferably, a fusion protein according to the present invention is used for medical treatment, if the infection to be treated or prevented is caused by multiresistant bacterial strains, in particular by strains resistant against one or more of the following antibiotics: streptomycin, tetracycline, cephalothin, gentamicin, cefotaxime, cephalosporin, ceftazidime or imipenem. Furthermore, a fusion protein according to the present invention can be used in methods of treatment by administering it in combination with conventional antibacterial agents, such as antibiotics, lantibiotics, bacteriocins or endolysins.

The present invention also relates to a pharmaceutical pack comprising one or more compartments, wherein at least one compartment comprises one or more fusion protein according to the present invention and/or one or more hosts transformed with a nucleic acid comprising a nucleotide sequence encoding a fusion protein according to the present invention or a composition according to the present invention.

In another aspect the present invention relates to a process of preparation of a pharmaceutical composition, said process comprising admixing one or more fusion protein according to the present invention and/or one or more hosts transformed with a nucleic acid comprising a nucleotide sequence encoding a fusion protein according to the present invention with a pharmaceutically acceptable diluent, excipient or carrier.

In an even further aspect the composition according to the present invention is a cosmetic composition. Several bacterial species can cause irritations on environmentally exposed surfaces of the patient's body such as the skin. In order to prevent such irritations or in order to eliminate minor manifestations of said bacterial pathogens, special cosmetic preparations may be employed, which comprise sufficient amounts of the fusion protein according to the present invention in order to degrade already existing or freshly settling pathogenic Gram-negative and/or Gram-positive bacteria.

In a further aspect the present invention relates to the fusion protein according to the present invention for use as diagnostic means in medicinal, food or feed or environmental diagnostics, in particular as a diagnostic means for the diagnostic of bacteria infection caused in particular by Gram-positive and/or Gram-negative bacteria. In this respect the fusion protein according to the present invention may be used as a tool to specifically degrade pathogenic bacteria, in particular Gram-positive and/or Gram-negative pathogenic bacteria. The degradation of the bacterial cells by the fusion protein according to the present invention can be supported by the addition of detergents like Triton X-100 or other additives which weaken the bacterial cell envelope like polymyxin B. Specific cell degradation is needed as an initial step for subsequent specific detection of bacteria using nucleic acid based methods like PCR, nucleic acid hybridization or NASBA (Nucleic Acid Sequence Based Amplification), immunological methods like IMS, immunofluorescence or ELISA techniques, or other methods relying on the cellular content of the bacterial cells like enzymatic assays using proteins specific for distinct bacterial groups or species e.g. β-galactosidase for enterobacteria, coagulase for coagulase positive strains.

In a further aspect the present invention relates to the use of the fusion protein according to the present invention for the treatment, removal, reduction or prevention of Gram-positive and/or Gram-negative bacterial contamination of foodstuff, of food processing equipment, of food processing plants, of surfaces coming into contact with foodstuff such as shelves and food deposit areas and in all other situations, where pathogenic, facultative pathogenic or other undesirable bacteria can potentially infest food material, of medical devices and of all kind of surfaces in hospitals and surgeries.

In a further aspect the present invention relates to a method for removal, reduction or prevention of Gram-positive and/or Gram-negative bacterial contamination of foodstuff, of food processing equipment, of food processing plants, of surfaces coming into contact with foodstuff such as shelves and food deposit areas and in all other situations, where pathogenic, facultative pathogenic or other undesirable bacteria can potentially infest food material, of medical devices and of all kind of surfaces in hospitals and surgeries comprising contacting said foodstuff, food processing equipment, food processing plants, surfaces coming into contact with foodstuff, medical devices or surfaces in hospitals with a fusion protein according to the present invention.

Another aspect of the present invention refers to a method for detecting a bacterium in a sample from a subject or in a food or feed, or environmental sample comprising contacting said sample with a fusion protein according to the present invention. The bacterium which is detected by a fusion protein of the present invention may be a Gram-positive and/or Gram-negative bacterium. A sample of a subject, in particular a human or animal, may be any body fluid, like blood serum and urine, as well as organs and tissues.

In particular, a fusion protein of the present invention may be used prophylactically as sanitizing agent. Said sanitizing agent may be used before or after surgery, or for example during hemodialysis. Moreover, premature infants and immunocompromised persons, or those subjects with need for prosthetic devices may be treated with a fusion protein according to the present invention. Said treatment may be either prophylactically or during acute infection. In the same context, nosocomial infections, especially by antibiotic resistant strains like Pseudomonas aeruginosa (FQRP), Acinetobacter species and Enterobacteriaceae such as E. coli, Salmonella, Shigella, Citrobacter, Edwardsiella, Enterobacter, Hafnia, Klebsiella, Morganella, Proteus, Providencia, Serratia, Yersinia species, Methicillin-resistant Staphylococcus aureus, Vancomycin-resistant Enterococcus faecalis, Vancomycin-resistant Enterococcus faecium, Streptococcus pneumoniae, Propionibacterium acnes, multidrug-resistant Mycobacterium tuberculosis, may be treated prophylactically or during acute phase with a fusion protein of the present invention. Therefore, a fusion protein according to the present invention may be used as a disinfectant also in combination with other ingredients useful in a disinfecting solution like detergents, tensids, solvents, antibiotics, lanthibiotics, or bacteriocins.

For the use of the fusion protein according to the present invention as a disinfectant e.g. in hospital, dental surgery, veterinary, kitchen or bathroom, the fusion protein can be prepared in a composition in form of e.g. a fluid, a powder, a gel, or an ingredient of a wet wipe or a disinfection sheet product. Said composition may additionally comprise suitable carrier, additives, diluting agents and/or excipients for its respective use and form, respectively,—but also agents that support the antimicrobial activity like EDTA or agents enhance the antimicrobial activity of the fusion proteins. The fusion protein may also be used with common disinfectant agents like, alcohols, aldehydes, oxidizing agents, phenolics, quaternary ammonium compounds or UV-light. For disinfecting for example surfaces, objects and/or devices the fusion protein can be applied on said surfaces, objects and/or devices. The application may occur for instance by wetting the disinfecting composition with any means such as a cloth or rag, by spraying, pouring. The fusion proteins may be used in varying concentration depending on the respective application and the, reaction time” intended to obtain full antimicrobial activity.

In a further aspect the present invention relates to the use of the fusion protein according to the present invention as a food additive.

Another aspect of the present invention is that the invention can be used like a tool box, i.e. any peptide stretch and antimicrobial peptide disclosed above may be fused to any endolysin, autolysin or bacteriocin disclosed herein. Thus, it is possible to combine the respective peptide stretch, which enables the binding of the fusion protein to the respective bacteria and the endolysin, autolysin or bacteriocin, which inhibit the growth of the respective bacteria. Consequently, it is possible to construct a suitable fusion protein for any bacteria which should be eliminated.

Further scope of applicability of the present invention will become apparent from the detailed description given hereinafter, however, it should be understood that the detailed description and specific examples, while indicating preferred embodiments of the invention, are given by way of illustration only, since various changes and modifications within the spirit and scope of the invention will become apparent to those skilled in the art from this detailed description. It is to be understood that both the foregoing general description and the following detailed description are exemplary and explanatory only and are not restrictive of the invention, as claimed.

The following examples explain the present invention but are not considered to be limiting. Unless indicated differently, molecular biological standard methods were used, as e.g., described by Sambrock et al., 1989, Molecular Cloning: A Laboratory Manual, 2nd edition, Cold Spring Harbor Laboratory Press, Cold Spring Harbor, N.Y.

### EXAMPLE 1

### N-Terminal Fusion of Antibacterial Peptide Tags to PK-Modified Endolysin (PK-OBPgpLys) of Pseudomonas putida Phage OBP

PK-OBPgpLys, the modified endolysin variant of P. putida phage OBP with an N-terminal polycationic peptide according to SEQ ID NO: 23, was N-terminally fused to natural antibacterial peptide tags, amphiphatic and hydrophobic peptide stretches (Table 4) to investigate its anti Gram-negative activity.

TABLE 4
List of antibacterial peptide tags which
were fused to PK-OBPgpLYS

| | Description + | Amino acid | |
|---|---|---|---|
| Tag | size | sequence | Reference |
| α4-helix of | Amphiphatic | PNRAKRVITTFRT | Matthews* |
| T4-lysozyme | helix | (SEQ ID NO: 68) | |
| | (13 aa) | | |
| Pentapeptide | Hydrophobic | FFVAP | Briers |
| (desianed) | (5 aa) | (SEQ ID NO: 66) | et al., |
| | | | 2008 |
| Walmagh1 | Hydrophobic | GFFIPAVILPSIAFLIVP | Walmagh 1 |
| (desianed) | (18 aa) | (SEQ ID NO: 65) | |
| Walmagh2 | Amphiphatic | GKPGWLIKKALVFKKLIRR | Walmagh 2 |
| (desianed) | helix | PLKRLA | |
| | (25 aa) | (SEQ ID NO: 69) | |
| Parasin 1 | Alpha-helical | KGRGKQGGKVRAKAKTRSS | Park, Y |
| | Peptide | (SEQ ID NO: 62) | et al., |
| | | | 1998** |
| Lycotoxin 1 | Amphiphatic | IWLTALKFLGKHAAKKLAK | Yan & |
| | helix | QQLSKL | Adams, |
| | (25 aa) | (SEQ ID NO: 61) | 1988*** |

*Matthews, B. W. and Remington, S. J. (1974). The three dimensional structure of the lysozyme from bacteriophage T4. Proc. Natl. Acad. Sci. USA, 71: 4178-4182
**In Yup Park, Chan Bae Park, Mi Sun Kim, Sun Chang Kim (1998). Parasin I, an antimicrobial peptide derived from histone H2A in the cat¢sh, Parasilurus asotus. FEBS Letters 437 258-262
***Yan, L and Adams, M. A. (1998). Lycotoxins, Antimicrobial Peptides from Venom of the Wolf Spider, Lycosa carolinensis J. Biol. Chem, 273:2059-2066.

### Methodology of Tag Modification of PK-OBPgpLys

To obtain the α4-, Walmagh 1-, Walmagh 2-, Lycotoxin1- and Parasin1-PK-OBPgplys ORFs, the corresponding peptide stretches (created by hybridization of primer pairs, see Table 5) were fused to the ORF which encodes for PK-OBPgpLYS using an adapted version of the Ligation Independent Cloning (LIC) technique. First, an unique Ec113611 restriction site (in bold) was inserted upstream of the PK modified OBPgpLys ORF by a tail PCR, with a specific designed 5′ primer encoding the restriction site (5′ GGAATGGGGAGCTCCTCCAAACGCAAGAAACGTAAGAAACGCAAAAAAAATAGCGAG AAT 3′; SEQ ID NO: 119) and the standard OBPgpLys reverse primer (5′ AACTATTCCGAGTGCTTTCTTTGT 3′; SEQ ID NO: 120) on purified genomic DNA of phage OBP. This extended fragment was then ligated into the pEXPSCT/TOPO® expression vector (Invitrogen, Carlsbad, Calif., USA) following the TA cloning protocol of the manufacturer. Pure plasmid was cut once (Ecl136II) and the hybridized peptide cassettes were inserted into the cut plasmid without a ligation step (LIC).

To fuse the PK-peptide stretch in front of the single tagged a4- and Pentapeptide-OBPgpLys ORFs a tail PCR with an extended 5′ primer encoding this PK peptide stretch (for PK-α4-OBPgpLys the 5′ primer is 5′ ATGGGATCCAAACGCAAGAAACGTAAGAAACGCAAAGGCTCCTCCCCGAACCGT GCA 3′; SEQ ID NO:121) and for PK-Pentapeptide-OBPgpLys the 5′ primer is 5′ ATGGGATCCAAACGCAAGAAACGTAAGAAACGCAAAGCCTCCTCCTTCTTCGTA GCA 3′ (SEQ ID NO: 122) and the standard 3′ OBPgpLys reverse primer was applied on purified pEXP5-CT/α4-OBPgpLys and pEXPS-CT/Pentapeptide-OBPgpLys plasmid DNA, respectively. Obtained PCR fragments were then recloned in the pEXPSCT/TOPO® expression vector. Correct insertion of the fragments in the expression vector was verified by sequencing analysis before introducing the construct into a suitable Escherichia coli BL21(DE3)pLysS expression strain.

TABLE 5
Used primer pairs for hybridization of antibacterial peptide
tags for N-terminal fusion to ORF encoding PK-OBPgpLYS

| Tag | forward primer | reverse primer |
|---|---|---|
| α4-helix | 5′TTGGAATGGGGAGCCCGAACCG | 5′GTTTGGAGGAGCCGGTACGGAAG |
| of T4- | TGCAAAACGTGTAATCA 3′ | GTGGTGATTACACGTT 3′ |
| lysozyme | (SEQ ID NO: 99) | (SEQ ID NO: 100) |
| Walmagh1 | 5′TTATGGGCTTCTTCATCCCGGCA | 5′GTTTGGAGGAGCCCGGTACGATC |
| (designed) | GTAATCCTGCCCTCCA 3′ | AGGAATGCGATGGAGGGCAGGAT 3′ |
| | (SEQ ID NO: 101) | (SEQ ID NO: 102) |
| Walmagh | 5′TTATGGGCAAACCGGGCTGGCT | 5′GTTTGGAGGAGCCTGCCAGTCTCT |
| 2 | GATCAAAAGGCACTGGTATTCAAG | TCAGCGGACGACGGATCAGTTTCTT |
| (designed) | A 3′ | GAATACCA 3′ |
| | (SEQ ID NO: 103) | (SEQ ID NO: 104) |
| Lycotoxin | 5′GGAATGGGGAGCATCTGGCTGA | 5′GTTTGGAGGAGCCCAGTTTGGATA |
| 1 | CCGCACTGAAATTCCTCGGCAAAC | ATTGCTGTTTTGCCAGTTTCTTTGCG |
| | ACGCCGCAA 3′ | GCGTGTT 3′ |
| | (SEQ ID NO: 105) | (SEQ ID NO: 106) |
| Parasin 1 | 5′TTGGAATGGGGAGCAAAGGCCG | 5′GTTTGGAGGAGCCTGAGGAACGG |
| | TGGCAAGCAGGGAGGCAAAGTAC | GTCTTTGCTTTTGCACGTACTTTGC |
| | GTG 3′ | 3′ |
| | (SEQ ID NO: 107) | (SEQ ID NO: 108) |

### Large Scale Recombinant Expression of Modified PK-OBPgpLYS Fusion Variants

Standard expression is performed in Lysogeny Broth (LB) in exponentially growing cells (OD₆₀₀ₙₘ=0.6) induced with 1 mM isopropyl-beta-D-thiogalactopyranoside. Expression parameters like temperature, time and expression strain varied on a protein specific basis in order to optimize the soluble expression levels of the modified endolysins (see Table 6).

For purification, cells from an expression culture (500-600 ml) are harvested (4500 rpm, 30 min, 4° C.) and resuspended in 1/25 volumes of lysis buffer (10 mM imidazole, 20 mM NaH₂PO₄, 0.5 M NaCl, pH 7.4). This suspension is frozen/thawed three times prior to sonication (8×30 s, amplitude 40% on a Vibra Cell™, Sonics, Dandurry, Conn., USA) and filtered through 0.45 and 0.22 μm Durapore membrane filters (Millipore, Billerica, Mass., USA). Purification of the His-tagged fusion protein was performed by a one-step protocol employing Ni²⁺-affinity chromatography (HisTrap HP 1 ml column, GE Healthcare, Buckinghamshire, UK) according to the manufacturer's instructions. The Ni²⁺ affinity chromatography is performed in 4 subsequent steps, all on room temperature:
1. Equilibration of the Histrap HP 1 ml column (GE Healthcare) with 10 column volumes of Washing Buffer (60 mM imidazole, 0.5 mM NaCl and 20 mM NaH₂PO₄—NaOH on pH 7.4) at a flow rate of 0.5 ml/min
2. Loading of the total lysate (with wanted endolysin) on the Histrap HP 1 ml column at a flow rate of 0.5 ml/min
3. Washing of the column with 15 column volumes of Washing Buffer at a flow rate of 1 ml/min
4. Elution of bounded endolysin from the column with 10 column volumes of Elution Buffer (500 mM imidazole, 5 mM NaCl and 20 mM NaH₂PO₄—NaOH on pH 7.4) at a flow rate of 0.5 ml/min

The wash buffer included a low imidazole concentration which varied on protein specific base to ensure higher purity of the protein (see Table 6). The total yields of recombinant proteins per liter E. coli expression culture is also shown in Table 6. The values were determined by spectrophotometric measurement of the protein concentration and the total volume of the purified stock solution at a wavelength of 280 nm. Purified stock solutions were at least 95% pure as determined visually on SDS-PAGE gels.

TABLE 6
Expression parameters and obtained protein yields per liter expression culture of N-
terminal modified PK-OBPgpLys variants

| | | Expression | Protein yield | Imidazole |
|---|---|---|---|---|
| Modified endolysin | Temperature/time | strain | (in mg/l) | (in mM) |
| α4-PK-OBPgpLys | 16° C./overnight | E.coli BL21 RIL | 0.56 | 60 |
| PK-α4-OBPgpLys | 16° C./overnight | E.coli BL21 RIL | 7.61 | 60 |
| Walmagh 1-PK-OBPgpLys | 16° C./overnight | E.coli BL21 RIL | 0.81 | 60 |
| Walmagh 2-PK-OBPgpLys | 16° C./overnight | E.coli BL21 RIL | 1.78 | 60 |
| Lycotoxin1-PK-OBPgpLys | 16° C./overnight | E.coli BL21 RIL | 0.23 | 60 |
| Parasin1-PK-OBPgpLys | 16° C./overnight | E.coli BL21 RIL | 8.08 | 60 |
| PK-pentapeptide-OBPgpLys | 16° C./overnight | E.coli BL21 RIL | 45.09 | 60 |

### In Vitro Antibacterial Activity and Host Range of Modified PK-OBPgpLys Fusion Variants

Exponential growing Gram-negative bacterial cells (OD600 nm=0.6) were 100-fold diluted to a final density of about 10⁶ cells/ml in 5 mM HEPES pH 7.4 and incubated for 30 minutes at room temperature without shaking with the different modified OBPgpLYS variants (in Elution Buffer in an end concentration of 1500 nM). After incubation cell suspensions and as a control untreated bacterial cells without any added modified OBPgpLYS variant were diluted three times (respectively 10⁵-10⁴-10³ cells/ml) in 1× concentrated PBS buffer and 100 μl of each dilution was plated out on LB-medium. The bacteria colonies on the agar plates were counted after an overnight incubation on 37° C. Based on the counted cell numbers the antibacterial activity as the relative inactivation in logarithmic units (=log 10N₀/Nᵢ with Nₒ=number of untreated cells (of the control) and Nᵢ=number of treated cells, both counted after incubation) is calculated (Table 7). The reduction in the number of bacteria colonies after treatment with fusion protein is indicative for an antimicrobial activity of the different modified OBPgpLYS variants, because the bacterial cells are lysed due to the treatment with the fusion proteins. All samples were replicated in threefold. Averages +/− standard deviations are represented.

TABLE 7
In vitro antibacterial activity of different N-terminal modified PK-OBPgpLYS fusion
variants on a range of exponential growing Gram-negative species without (A) and with
(B) extra addition of the outer membrane permeabilizer EDTA (0.5 mM). Initial density is
10⁶ cells/ml and incubation proceeds for 30 minutes without shaking at RT. Average
log reduction values are shown together with standard deviation (stdev).

| | P. aeruginosa | | | | | | S. typhimurium | |
|---|---|---|---|---|---|---|---|---|
| | PAO1p | | P. putida G1 | | E. coli XL1 blue | | LT2 | |
| Fusion protein | average | stdev | average | stdev | average | stdev | average | stdev |

A

| | | | | | | | | |
|---|---|---|---|---|---|---|---|---|
| α4-PK-OBPgpLys | 0.59 | 0.02 | 0.02 | 0.13 | 0.55 | 0.06 | 0.22 | 0.03 |
| PK-α4-OBPgpLys | 1.10 | 0.05 | 0.01 | 0.04 | 0.54 | 0.17 | 0.23 | 0.04 |
| Walmagh1-PK- | | | | | | | | |
| OBPgpLYS | 0.20 | 0.09 | 0.29 | 0.22 | 1.46 | 0.08 | 0.12 | 0.03 |
| Walmagh2-PK- | | | | | | | | |
| OBPgplys | 0.57 | 0.10 | 0.00 | 0.04 | 1.68 | 0.12 | 0.17 | 0.02 |
| Lycotoxin1-PK- | | | | | | | | |
| OBPgpLYS | 0.08 | 0.05 | 0.56 | 0.10 | 0.41 | 0.04 | 0.17 | 0.07 |
| Parasin1-PK-OBPgpLys | 0.75 | 0.03 | 0.16 | 0.04 | 0.70 | 0.09 | 0.27 | 0.11 |
| PK-Pentapeptide- | | | | | | | | |
| OBPgplys | 0.95 | 0.08 | 0.80 | 0.04 | 1.69 | 0.17 | 0.22 | 0.06 |

B

| | | | | | | | | |
|---|---|---|---|---|---|---|---|---|
| EDTA | 1.82 | +/−0.17 | 0.65 | +/−0.35 | 0.85 | +/−0.16 | 0.08 | +/−0.01 |
| α4-PK- | | | | | | | | |
| OBPgpLys + EDTA | 5.23 | +/−0.03 | 2.98 | +/−0.16 | 0.58 | +/−0.13 | 0.35 | +/−0.11 |
| PK-α4- | | | | | | | | |
| OBPgpLys + EDTA | 5.07 | +/−0.21 | 2.97 | +/−0.09 | 0.86 | +/−0.12 | 0.29 | +/−0.06 |
| Walmagh1-PK- | | | | | | | | |
| OBPgpLYS + EDTA | 3.66 | +/−0.12 | 3.22 | +/−0.21 | 1.70 | +/−0.07 | 0.17 | +/−0.07 |
| Walmagh2-PK- | | | | | | | | |
| OBPgplys + EDTA | 3.91 | +/−0.09 | 2.60 | +/−0.12 | 1.86 | +/−0.10 | 0.25 | +/−0.04 |
| Lycotoxin1-PK- | | | | | | | | |
| OBPgpLYS + EDTA | 3.73 | +/−0.37 | 2.26 | +/−0.10 | 0.90 | +/−0.24 | 0.29 | +/−0.06 |
| Parasin1-PK- | | | | | | | | |
| OBPgpLys + EDTA | 4.92 | +/−0.03 | 2.32 | +/−0.08 | 1.00 | +/−0.14 | 0.28 | +/−0.05 |
| PK-Pentapept- | | | | | | | | |
| OBPgpLys + EDTA | 4.00 | +/−0.06 | 3.24 | +/−0.26 | 2.12 | +/−0.33 | 0.42 | +/−0.05 |

### EXAMPLE 2

### Antimicrobial Activity of Fusion Proteins of Endolysin (PK-OBPgpLys) of Pseudomonas putida Phage OBP with Two Peptide Stretches in Comparison to Fusion Protein of Endolysin (PK-OBPgpLys) of Pseudomonas putida Phage OBP with Only One Peptide Stretch

Methodology of Production of OBPgpLys with One Peptide Stretch

Except for the pentapeptide, all peptide stretches were fused to the ORF which encodes for the OBPgpLYS derivative using an adapted version of the Ligation Independent Cloning (LIC) as e.g. described in Berrow et al. 2007. Here fore, an unique Ec113611 restriction site was inserted in front of the WT endolysin gene by a tail PCR with a specific designed 5′ primer (5′-GGAATGGGGAGCTCCTCCAAAAATAGCGAGAAG-3′; SEQ ID NO: 123) and the standard OBPgpLys derivative reverse primer (5′-AACTATTCCGTGTGCTTTCTTTGT-3′; SEQ ID NO:124) on pure genomic DNA of phage OBP. This extended fragment was then ligated in the pEXPSCT/TOPO® expression vector (Invitrogen, Carlsbad, Calif., USA) by following the TA cloning protocol of the manufacturer. Pure plasmid was cutted once in an Ec113611 restriction digest and hybridized peptide cassettes (created by hybridization of primer pairs, see Table 8) were inserted into the cutted plasmid without a necessary ligation step (LIC). For the N-terminal pentapeptide tag fusion a tail PCR with an extended 5′ primer which encodes for this pentapeptide (5′-ATGGGATCCTTCTTCGTAGCA CCGGGCTCCTCCAAAAATAGCGAGAAG-3′; SEQ ID NO: 125) and the standard OBPgpLys derivative reverse primer (5′-AACTATTCCGTGTGCTTTCTTTGT-3′; SEQ ID NO: 126) was applied on phage OBP genomic DNA. Correct insertion of the fragments in the expression vector was verified by sequencing analysis before introducing the construct into a suitable Escherichia coli BL21(DE3)pLysS expression strain.

TABLE 8
Used primer pairs for hybridization of antibacterial
peptide tags to ORF encoding the OBPgpLys derivative

| Tag | forward primer | reverse primer |
|---|---|---|
| α4-helix | 5′TTGGAATGGGGAGCCCGAACCGTG | 5′TATTTTTGGAGGAGCCGGTACGGAAG |
| of T4- | CAAAACGTGTAATCA 3′ | GTGGTGATTACACGTT 3′; |
| lysozyme | (SEQ ID NO: 109) | (SEQ ID NO: 110) |
| Walmagh1 | 5′TTATGGGCTTCTTCATCCCGGCAGT | 5′TATTTTTGGATCTGCCGCCCGGTACGA |
| (designed) | AATCCTGCCCTCCA 3′ | TCAGGAATGCGATGGAGGGCAGGATT |
| | (SEQ ID NO: 111) | 3′; |
| | | (SEQ ID NO: 112) |
| Walmagh 2 | 5′TTATGGGCAAACCGGGCTGGCTGA | 5′TATTTTTGGATCTGCCGCCTGCCAGTC |
| (designed) | TCAAAAGGCACTGGTATTCAAGA 3′ | TCTTCAGCGGACGACGGATCAGTTTCTT |
| | (SEQ ID NO: 113) | GAATACCAG 3′; |
| | | (SEQ ID NO: 114) |
| Parasin 1 | 5′TTGGAATGGGGAGCAAAGGCCGTG | 5′TATTTTTGGAGGAGCCTGAGGAACGG |
| | GCAAGCAGGGAGGCAAAGTACGTG 3′ | GTCTTTGCTTTTGCACGTACTTTGC 3′; |
| | (SEQ ID NO: 117) | (SEQ ID NO: 118) |
| Lycotoxin 1 | 5′GGAATGGGGAGCATCTGGCTGACC | 5′TATTTTTGGAGGAGCCCAGTTTGGATA |
| | GCACTGAAATTCCTCGGCAAACACGC | ATTGCTGTTTTGCCAGTTTCTTTGCGGC |
| | CGCAA 3′ | GTGTT 3′; |
| | (SEQ ID NO: 115) | (SEQ ID NO: 116) |

The production of OBPgpLys with two peptide stretches is described above in Example 1. Moreover, the purification of the fusion proteins and the antimicrobial activity assay was performed as described already in Example 1. The results for the antimicrobial activity assay are given in Table 9.

TABLE 9
In vitro antibacterial activity of OBPgpLYS fusion variants with one or two peptide
stretcthes on a range of exponential growing Gram-negative species with extra addition
of the outer membrane permeabilizer EDTA (0.5 mM). Initial density is 10⁶ cells/ml
and incubation proceeds for 30 minutes without shaking at RT. Average log reduction
values are shown.

| | | | | Salmonella |
|---|---|---|---|---|
| | P. aeruginosa | P. putida | | typhimurium |
| Fusion protein | PAO 1 p | G1 | E. coli. X1-1 | LT2 |
| α4-OBPgpLys | ++ | ++ | ++ | + |
| α4-PK-OBPgpLys | +++ | ++ | + | + |
| PK-α4-OBPgpLys | +++ | ++ | ++ | + |
| Pentapeptide- | ++ | +++ | ++ | + |
| OBPgpLys | | | | |
| PK-Pentapeptide- | +++ | +++ | ++ | + |
| OBPgplys | | | | |
| Walmagh2-OBPgpLys | ++ | ++ | ++ | + |
| Walmagh2-PK- | +++ | ++ | ++ | + |
| OBPgplys | | | | |
| Parasin1-OBPgpLys | +++ | +++ | +++ | ++ |
| Parasin1-PK- | +++ | ++ | ++ | + |
| OBPgpLys | | | | |
| Lycotoxin1- | ++ | +++ | ++ | + |
| OBPgpLys | | | | |
| Lycotoxin1-PK- | +++ | ++ | ++ | + |
| OBPgpLys | | | | |
| OBPgpLYS | + | + | + | + |

Abbreviations: +: 1 log; ++: 2-3 log; +++: 4 or more logs

### EXAMPLE 3

### Antimicrobial Activity of Fusion Proteins of Pseudomonas aeruginosa Bacteriophage Endolysins KZ144 (Phage phiKZ) and EL188 (Phage EL) with Two Peptide Stretches in Comparison to Fusion Protein of Endolysin of Pseudomonas aeruginosa Phage KZ144 and EL188 with Only One or without Peptide Stretch

The purification of the fusion proteins and the antimicrobial activity assay was performed as described in Example 1. The results for the antimicrobial activity assay are given in Table 10.

TABLE 10
In vitro antibacterial activity of KZ144 and EL188 fusion
variants with one or two peptide stretches on a range of
exponential growing Pseudomonas bacteria with extra
addition of the outer membrane permeabilizer EDTA (0.5 mM).
Initial density is 10⁶ cells/ml and incubation proceeds for 30
minutes without shaking at RT. Average log reduction values
are shown.

| Protein | Pseudomonas |
|---|---|
| KZ144 (SEQ ID NO: 18) | +/− |
| KZ144-PK (SEQ ID NO: 148) | + |
| KZ144-SMAP29 (SEQ ID NO: 149) | ++ |
| KZ144-SMAP29-PK (SEQ ID NO: 140) | +++ |
| EL188 (SEQ ID NO: 7) | +/− |
| EL188-SMAP29 (SEQ ID NO: 150) | + |
| EL188-PK (SEQ ID NO: 151) | + |
| EL188-PK-SMAP29 (SEQ ID NO: 141) | +++ |

Abbreviations: −: no log reduction; +/−: <1 log; +: 1 log; ++: 2-3 log; +++: 4 or more logs

### EXAMPLE 4

### Antimicrobial Activity of Fusion Proteins of Endolysin STM0016 with Two Peptide Stretches in Comparison to Fusion Protein of Endolysin STM0016 with Only One or without Peptide Stretch

The purification of the fusion proteins and the antimicrobial activity assay was performed as described in Example 1. The results for the antimicrobial activity assay are given in Table 11.

TABLE 11
In vitro antibacterial activity of STM0016 fusion variants with
one or two peptide stretches on a range of exponential growing
Salmonella bacteria with extra addition of the outer membrane
permeabilizer EDTA (0.5 mM). Initial density is 10⁶ cells/ml and
incubation proceeds for 30 minutes without shaking at RT.
Average log reduction values are shown.

| Protein | Salmonella |
|---|---|
| STM0016 (SEQ ID NO: 15) | − |
| STM0016-SMAP29 (SEQ ID NO: 152) | + |
| STM0016-LL37 (SEQ ID NO: 153) | + |
| STM0016-Melittin (SEQ ID NO: 154) | +/− |
| STM0016-LL37-SMAP29 (SEQ ID NO: 142) | +++ |
| STM0016-SMAP29-Melittin (SEQ ID NO: 143) | ++ |

Abbreviations: −: no log reduction; +/−: <1 log; +: 1 log; ++: 2-3 log; +++: 4 or more logs

### EXAMPLE 5

### Antimicrobial Activity of Fusion Proteins of Endolysin Lysostaphin with Two Peptide Stretches in Comparison to Fusion Protein of Endolysin Lysostaphin with Only One or without Peptide Stretch

The purification of the fusion proteins and the antimicrobial activity assay was performed as described in Example 1. The results for the antimicrobial activity assay are given in Table 12.

TABLE 12
In vitro antibacterial activity of Lysostaphin fusion variants with
one or two peptide stretches on a range of exponential growing
Staphylococcus bacteria with extra addition of the outer membrane
permeabilizer EDTA (0.5 mM). Initial density is 10⁶ cells/ml and
incubation proceeds for 30 minutes without shaking at RT.
Average log reduction values are shown.

| Protein | Staphylococcus |
|---|---|
| Lysostaphin (SEQ ID NO: 4) | + |
| Lysostaphin-Sarcotoxin IA (SEQ ID NO: 155) | ++ |
| Lysostaphin-SMAP29 (SEQ ID NO: 156) | ++ |
| Sarcotoxin IA-Lysostaphin (SEQ ID NO: 157) | ++ |
| SMAP29-Lysostaphin (SEQ ID NO: 158) | ++ |
| Lysostaphin-SMAP29-Sarcotoxin IA (SEQ ID NO: 144) | +++ |
| Sarcotoxin IA-SMAP29-Lysostaphin (SEQ ID NO: 131) | +++ |

Abbreviations: −: no log reduction; +/−: <1 log; +: 1 log; ++: 2-3 log; +++: 4 or more logs

### EXAMPLE 6

### Antimicrobial Activity of Fusion Proteins of Endolysin Cp11 with Two Peptide Stretches in Comparison to Fusion Protein of Endolysin Cp11 with Only One or without Peptide Stretch

The purification of the fusion proteins and the antimicrobial activity assay was performed as described in Example 1. The results for the antimicrobial activity assay are given in Table 13.

TABLE 13
In vitro antibacterial activity of Cpl1 fusion variants with
one or two peptide stretches on a range of exponential growing
Streptococcus bacteria with extra addition of the outer membrane
permeabilizer EDTA (0.5 mM). Initial density is 10⁶ cells/ml and
incubation proceeds for 30 minutes without shaking at RT.
Average log reduction values are shown.

| Protein | Streptococcus |
|---|---|
| Cpl1 (SEQ ID NO: 1) | + |
| Cpl1-Indolicidin (SEQ ID NO: 159) | ++ |
| Cpl1-Pseudin1 (SEQ ID NO: 160) | ++ |
| Cpl1-Pseudin1-Indolicidin (SEQ ID NO: 145) | +++ |

Abbreviations: −: no log reduction; +/−: <1 log; +: 1 log; ++: 2-3 log; +++: 4 or more logs

### EXAMPLE 7

### Antimicrobial Activity of Fusion Proteins of Endolysin Ply511 with Two Peptide Stretches in Comparison to Fusion Protein of Endolysin Ply511 with Only One or without Peptide Stretch

The purification of the fusion proteins and the antimicrobial activity assay was performed as described in Example 1. The results for the antimicrobial activity assay are given in Table 14.

TABLE 14
In vitro antibacterial activity of Ply511 fusion variants with one or
two peptide stretches on a range of exponential growing Listeria
bacteria with extra addition of the outer membrane permeabilizer
EDTA (0.5 mM). Initial density is 10⁶ cells/ml and incubation
proceeds for 30 minutes without shaking at RT. Average log
reduction values are shown.

| Protein | Listeria |
|---|---|
| P1y511 (SEQ ID NO: 2) | + |
| P1y511-Magainin (SEQ ID NO: 161) | ++ |
| P1y511-BuforinII (SEQ ID NO: 162) | ++ |
| P1y511-BuforinII-Magainin (SEQ ID NO: 146) | +++ |

Abbreviations: −: no log reduction; +/−: <1 log; +: 1 log; ++: 2-3 log; +++: 4 or more logs

### EXAMPLE 8

### Antimicrobial Activity of Fusion Proteins of Endolysin LysK with Two Peptide Stretches in Comparison to Fusion Protein of Endolysin LysK with Only One or without Peptide Stretch

The purification of the fusion proteins and the antimicrobial activity assay was performed as described in Example 1. The results for the antimicrobial activity assay are given in Table 15.

TABLE 15
In vitro antibacterial activity of LysK fusion variants with one or
two peptide stretches on a range of exponential growing
Staphylococcus bacteria with extra addition of the outer
membrane permeabilizer EDTA (0.5 mM). Initial density is 10⁶
cells/ml and incubation proceeds for 30 minutes without shaking
at RT. Average log reduction values are shown.

| Protein | Staphylococcus |
|---|---|
| LysK (SEQ ID NO: 3) | + |
| PK2-LysK (SEQ ID NO: 163) | ++ |
| Cecropin A (A. aegyptii)-LysK (SEQ ID NO: 164) | ++ |
| PK2-Cecropin A (A. aegyptii)-LysK (SEQ ID NO: 147) | +++ |

Abbreviations: −: no log reduction; +/−: <1 log; +: 1 log; ++: 2-3 log; +++: 4 or more logs

### EXAMPLE 9

### Antimicrobial Activity of Different Peptide Stretches

Exponential P. aeruginosa PAO1p cells (Burn wound isolate, Queen Astrid Hospital, Brussels; Pirnay J P et al. (2003), http://www.ncbi.nlm.nih.gov/pubmed/12624051?ordinalpos=3&itool=EntrezSystem2.PEntre z.Pubmed.Pubmed_ResultsPanel.Pubmed_DefaultReportPanel.Pubmed_RVDocSum J Clin Microbiol., 41(3):1192-1202) were washed in 10 mM HEPES, pH 7.4 and diluted 1:10 in 10 mM HEPES, 0.5 mM EDTA, pH 7.4. Bacteria were incubated at room temperature with the respective peptide, polycationic peptide named PK according to SEQ ID NO: 23, the polycationic peptide PK2 according to SEQ ID NO: 34 or the antimicrobial peptide named SMAP-29 according to SEQ ID NO: 45 in buffer (15 mM HEPES, 250 mM NaCl, 250 μM EDTA; pH 7.4) in the concentrations 0.3 mmol/L, 3 mmol/L, 30 mmol/L and 150 mmol/L for the polycationic peptides and 3 mmol/L for the antimicrobial peptide. After 30 minutes cell suspensions were serial diluted (1:10) and plated on LB. Additionally, a negative control was plated using buffer (15 mM HEPES, 250 mM NaCl, 250 μM EDTA; pH 7.4). The bacteria colonies on the agar plates were counted after an overnight incubation at 37° C. Based on the counted cell numbers the antibacterial activity as the relative inactivation in logarithmic units (=log 10N₀/Nᵢ with N₀=number of untreated cells (of the control) and Nᵢ=number of treated cells, both counted after incubation) is calculated. The reduction in the number of bacteria colonies after treatment with a peptide stretch is indicative for an antimicrobial activity of the peptide stretch, because the bacterial cells are lysed due to the treatment with the peptide stretch. All samples were replicated in four fold.

Both polycationic peptides PK and PK2 showed increasing antimicrobial activity against P. aeruginosa dependent on the used concentration of the peptide. PK2 showed higher antimicrobial activity against P. aeruginosa than PK. Moreover, PK2 used in a concentration of 150 mmol/L showed same antimicrobial activity against P. aeruginosa as SMAP-29 used in a concentration of 3 mmol/L. For both PK and PK2 more peptide has to be used to achieve the same antimicrobial activity against P. aeruginosa as SMAP-29. Usage of equimolar concentrations of PK or PK2 (3 μM/L) show no or only minor antimicrobial effects compared to AMP or Artilysin.

1. A fusion protein comprising an enzyme having the activity of degrading the cell wall of Gram-negative bacteria and/or Gram-positive bacteria and at least two peptide stretches fused to the enzyme at the N- or C-terminus, wherein the peptide stretches are distinct and selected from the group of synthetic amphiphatic peptide, synthetic cationic peptide, synthetic polycationic peptide, synthetic hydrophobic peptide, or naturally occurring antimicrobial peptide, like sushi peptide and defensin.

2. The fusion protein according to claim 1, wherein the enzyme is an endolysin, autolysin or bacteriocin.

3. The fusion protein according to claim 1, wherein the synthetic peptide or naturally occurring peptide has a length of 6 to 39 amino acid residues.

4. The fusion protein according to claim 1, wherein the Gram-negative bacteria are selected from the group consisting of
Enterobacteriaceae,
in particular Escherichia, Salmonella, Shigella, Citrobacter, Edwardsiella, Enterobacter, Hafnia, Klebsiella, Morganella, Proteus, Providencia, Serratia, and Yersinia,
Pseudomonadaceae,
in particular Pseudomonas, Burkholderia, Stenotrophomonas, Shewanella, Sphingomonas and Comamonas,
Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae,
in particular Treponema and Borrelia,
Leptospiraceae, Campylobacter, Helicobacter, Spirillum, Streptobacillus, Bacteroidaceae,
in particular Bacteroides, Fusobacterium, Prevotella and Porphyromonas, and
Acinetobacter,
in particular A. baumanii; and
wherein the Gram-positive bacteria are selected from the group consisting of Listeria monocytogenes, Staphylococcus aureus, Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium tuberculosis, Corynebacterium diphteriae, Mycoplasma pneumoniae, and Actinomyces.

5. The fusion protein according to claim 1, wherein the enzyme exhibits an amino acid sequence according to SEQ ID NO: 1 to 18.

6. The fusion protein according to claim 1, wherein the antimicrobial peptide exhibits an amino acid sequence according to SEQ ID NO: 44 to 62 and 78 to 98, or wherein the sushi peptide exhibits an amino acid sequence according to SEQ ID NO: 63; or wherein the hydrophobic peptide exhibits an amino acid sequence according to SEQ ID NO: 65 and/or 66; or wherein the amphiphatic peptide exhibits an amino acid sequence according to SEQ ID NO: 64, 68 and/or 69.

7. The fusion protein according to claim 1, wherein said fusion protein exhibits an amino acid sequence according to SEQ ID NO: 70 to 77 and 127 to 147.

8. An isolated nucleic acid molecule encoding a fusion protein according to claim 1.

9. A vector comprising the nucleic acid molecule according to claim 8.

10. A host cell comprising the nucleic acid molecule according to claim 8.

11. The host cell according to claim 10, wherein the cell is a bacterial cell or a yeast cell.

12. The fusion protein according to claim 1, formulated as a medicament, diagnostic, cosmetic substance, a disinfectant or food additive.

13. (canceled)

14. The method according to claim 18, wherein the disorder, disease or condition is caused by Gram-negative bacteria of bacterial groups, families, genera or species comprising strains pathogenic for humans or animals, in particular Enterobacteriaceae (Escherichia, especially E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (Pseudomonas, especially P. aeruginosa, Burkholderia, Stenotrophomonas, Shewanella, Sphingomonas, Comamonas), Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (Treponema and Borrelia), Leptospiraceae, Campylobacter, Helicobacter, Spirillum, Streptobacillus, Bacteroidaceae (Bacteroides, Fusobacterium, Prevotella, Porphyromonas), Acinetobacter, especially A. baumanii or by Gram-positive bacteria of bacterial groups, families, genera or species comprising strains pathogenic for humans or animals in particular Listeria monocytogenes, Staphylococcus aureus, Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium tuberculosis, Corynebacterium diphteriae, Mycoplasma pneumoniae, Actinomyces.

15. A pharmaceutical composition comprising a fusion protein according to claim 1.

16. A method for the production of a fusion protein comprising culturing a host cell according to claim 10 under conditions supporting the expression of said fusion protein.

17. A method of treating a disorder, disease or condition caused by Gram-negative and/or Gram-positive bacteria in a subject comprising administering to said subject a fusion protein according to claim 1.

18. The method according to claim 17, wherein the disorder, disease or condition is a bacterial infection caused by Gram-positive and/or Gram-negative bacteria.

19. A method for the removal, reduction and/or prevention of Gram-negative and/or Gram-positive bacterial contamination of foodstuff, of food processing equipment, of food processing plants, of surfaces coming into contact with foodstuff, of medical devices, of surfaces in hospitals and surgeries comprising contacting said foodstuff, food processing equipment, food processing plants, surfaces coming into contact with foodstuff, medical devices, or surfaces in hospitals with a fusion protein according to claim 1.

20. A method for detecting a bacterium in a sample from a subject or in a food or feed, or environmental sample comprising contacting said sample with a fusion protein according to claim 1.

21. The use of the fusion protein according to claim 1 for the treatment or prevention of Gram-negative and/or Gram-positive bacterial contamination of foodstuff, of food processing equipment, of food processing plants, of surfaces coming into contact with foodstuff, of medical devices, of surfaces in hospitals and surgeries.

22. The use of the fusion protein according to claim 1 as a diagnostic means in medicinal, food or feed or environmental diagnostics.


SEQUENCE LISTING (164 sequences)

SEQ ID NO: 1 - protein, 339 aa, Streptococcus pneumoniae Phage Cpl-1
MVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVA

SEQ ID NO: 2 - protein, 341 aa, Listeria monocytogenes Phage A511
MVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKK

SEQ ID NO: 3 - protein, 495 aa, Staphylococcus aureus Phage K
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 4 - protein, 245 aa, Staphylococcus simulans
MAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 5 - protein, 286 aa, Propionibacterium acnes
MVRYIPAAHHSAGSNNPVNRVVIHATCPDVGFPSASRKGRAVSTANYFASPSSGGSAHYVCDIGETVQCLSESTIGWHAPPNPHSLGIEICADGGSHASFRVPGHAYTREQWLDPQVWPAVERAAVLCRRLCDKYNVPKRKLSAADLKAGRRGVCGHVDVTDAWHQSDHDDPGPWFPWDKFMAVVNGGSGDSGELTVADVKALHDQIKQLSAQLTGSVNKLHHDVGVVQVQNGDLGKRVDALSWVKNPVTGKLWRTKDALWSVWYYVLECRSRLDRLESAVNDLKK

SEQ ID NO: 6 - protein, 260 aa, Pseudomonas aeruginosa phage KZ
MKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 7 - protein, 292 aa, Pseudomonas aeruginosa phage EL
MNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGLDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISY

SEQ ID NO: 8 - protein, 181 aa, unknown
  Salmonella endolysin
MKPKDEIFDEILGKEGGYVNHPDDKGGPTKWGITEKVARAHGYRGDMRNLTRGQALEILETDYWYGPRFDRVAKASPDVAAELCDTGVNMGPSVAAKMLQRWLNVFNQGGRLYPDMDTDGRIGPRTLNALRVYLEKRGKDGERVLLVALNCTQGERYLELAEKREADESFVYGWMKERVLI

SEQ ID NO: 9 - protein, 163 aa, Enterobacteria phage T4
MNIFEMLRIDEGLRLKIYKDTEGYYTIGIGHLLTKSPSLNAAKSELDKAIGRNCNGVITKDEAEKLFNQDVDAAVRGILRNAKLKPVYDSLDAVRRCALINMVFQMGETGVAGFTNSLRMLQQKRWDEAAVNLAKSRWYNQTPNRAKRVITTFRTGTWDAYKN

SEQ ID NO: 10 - protein, 280 aa, Acinetobacter baumannii phage
MEYDMILKFGSKGDAVATLQKQLAKMGYKGVKDKPLSVDGHFGESTEFAVIQLQRKFGLVADGKVGDKTRQALAGDSVSKFLKDEDYKKAAIRLKVPELVIRVFGAVEGLGVGFLPNGKAKILFERHRMYFYLCQALGKTFANSQVKITPNIVNTLTGGYKGDAAEYTRLSMAINIHKESALMSTSWGQFQIMGENWKDLGYSSVQEFVDQQQLNEGNQLEAFIRFIEWKPGLLEALRKQDWDTVFTLYNGKNYKKLGYQAKFQKEWDHLEPIYREKTAA

SEQ ID NO: 11 - protein, 152 aa, E. coli phage K1F
MVSKVQFNPRSRTDAIFVHCSATKPEMDIGVETIRMWHKQQAWLDVGYHFIIKRDGTVEEGRPVNVVGSHVKDWNSRSVGVCLVGGINAKGQFEANFTPAQMNSLRNKLDDLKVMYPQAEIRAHHDVAPKACPSFDLQRWLSTNELVTSDRG

SEQ ID NO: 12 - protein, 328 aa, Pseudomonas putida phage
MKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 13 - protein, 165 aa, unknown
  Salmonella endolysin
MPVINTHQNIAAFLDMLAYSEGTANHPLTKNRGYDVIVTGFDGSPEIFTDYSDHPFAHGRPPKVFNRRGEKSTASGRYQQLYIFWPHYKKQLALPDFSPLSQDKLAIQLIRERGAIDDIRAGRIERAVSRCRNIWASLPGAGYGQREHSLEKLVTVWRTAGGVMA

SEQ ID NO: 14 - protein, 165 aa, E.coli Phage P2
MPVINTHQNIAAFLDMLAVSEGTANHPLTKNRGYDVIVTGLDGKPEIFTDYSDHPFAHGRPAKVFNRRGEKSTASGRYQQLYLFWPHYRKQLALPDFSPLSQDRLAIQLIRERGALDDIRAGRIERAISRCRNIWASLPGAGYGQREHSLEKLVTVWRTAGGVPA

SEQ ID NO: 15 - protein, 177 aa, Salmonella typhimurium phage
MNPIIDGIIALEGGYVFNPKDKGGATHWGITEATARAHGYAGDMRDLTHAEAYAILEEDYWIKPGFDVISTLSWPVSFELCDAAVNIGAYHPSAWLQRWLNVFNHEGKRYPDIHVDGNIGPRTLAALEHYLAWRGQEGEAVLVKALNCSQGTYYLNVAEKNHNNEQFIYGWIKNRVT

SEQ ID NO: 16 - protein, 208 aa, E. coli Phage N4
MAISKKKVGGVGGVIAAIIAAVFAVEGGYVNDPKDPGGETNHGVTIQVAQKHKQELESMYNWDGSMKNLTQEMASSIYYNDYILKPGFVKFADVSPAVTEKLVDAGVNTGPARPSRWLQESLNAFSRNGKDYPKIQVDGKVGSGTLSAYKSLQNKRGKVEACKLILKSLDGKQLNYYLSLNMPEYTTGWIANRIGNVPLERCNEDIVN

SEQ ID NO: 17 - protein, 185 aa, unknown
  N4-gp61 trunc.
MVEGGYVNDPKDPGGETNHGVTIQVAQKHKQELESMYNWDGSMKNLTQEMASSIYYNDYILKPGFVKFADVSPAVTEKLVDAGVNTGPARPSRWLQESLNAFSRNGKDYPKIQVDGKVGSGTLSAYKSLQNKRGKVEACKLILKSLDGKQLNYYLSLNMPEYTTGWIANRIGNVPLERCNEDIVN

SEQ ID NO: 18 - protein, 259 aa, Pseudomonas aeruginosa phage KZ
KVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 19 - protein, 6 aa, artificial
  synthetic sequence
KRKKRK

SEQ ID NO: 20 - protein, 5 aa, artificial
  synethtic sequence
KRXKR

SEQ ID NO: 21 - protein, 5 aa, artificial
  synthetic sequence
KRSKR

SEQ ID NO: 22 - protein, 5 aa, artificial
  synthetic sequence
KRGSG

SEQ ID NO: 23 - protein, 9 aa, artificial
  synthetic sequence
KRKKRKKRK

SEQ ID NO: 24 - protein, 9 aa, artificial
  synthetic sequence
RRRRRRRRR

SEQ ID NO: 25 - protein, 8 aa, artificial
  synthetic sequence
KKKKKKKK

SEQ ID NO: 26 - protein, 10 aa, artificial
  synthetic sequence
KRKKRKKRKK

SEQ ID NO: 27 - protein, 12 aa, artificial
  synthetic sequence
KRKKRKKRKKRK

SEQ ID NO: 28 - protein, 14 aa, artificial
  synthetic sequence
KRKKRKKRKKRKKR

SEQ ID NO: 29 - protein, 16 aa, artificial
  synthetic sequence
KKKKKKKKKKKKKKKK

SEQ ID NO: 30 - protein, 18 aa, artificial
  synthetic sequence
KRKKRKKRKKRKKRKKRK

SEQ ID NO: 31 - protein, 19 aa, artificial
  synthetic sequence
KRKKRKKRKKRKKRKKRKK

SEQ ID NO: 32 - protein, 19 aa, artificial
  synthetic sequence
RRRRRRRRRRRRRRRRRRR

SEQ ID NO: 33 - protein, 19 aa, artificial
  synthetic sequence
KKKKKKKKKKKKKKKKKKK

SEQ ID NO: 34 - protein, 20 aa, artificial
  synthetic sequence
KRKKRKKRKRSKRKKRKKRK

SEQ ID NO: 35 - protein, 21 aa, artificial
  synthetic sequence
KRKKRKKRKRSKRKKRKKRKK

SEQ ID NO: 36 - protein, 21 aa, artificial
  synthetic sequence
KRKKRKKRKKRKKRKKRKKRK

SEQ ID NO: 37 - protein, 22 aa, artificial
  synthetic sequence
KRKKRKKRKRGSGKRKKRKKRK

SEQ ID NO: 38 - protein, 24 aa, artificial
  synthetic sequence
KRKKRKKRKRGSGSGKRKKRKKRK

SEQ ID NO: 39 - protein, 25 aa, artificial
  synthetic sequence
KRKKRKKRKKRKKRKKRKKRKKRKK

SEQ ID NO: 40 - protein, 31 aa, artificial
  synthetic sequence
KRKKRKKRKRSKRKKRKKRKRSKRKKRKKRK

SEQ ID NO: 41 - protein, 38 aa, artificial
  synthetic sequence
KRKKRKKRKRGSGSGKRKKRKKRKGSGSGKRKKRKKRK

SEQ ID NO: 42 - protein, 39 aa, artificial
  synthetic sequence
KRKKRKKRKKRKKRKKRKKRKKRKKRKKRKKRKKRKKRK

SEQ ID NO: 43 - protein, 42 aa, artificial
  synthetic sequence
KRKKRKKRKRSKRKKRKKRKRSKRKKRKKRKRSKRKKRKKRK

SEQ ID NO: 44 - protein, 37 aa, Homo sapiens
LLGDFFRKSKEKIGKEFKRIVQRIKDFLRNLVPRTES

SEQ ID NO: 45 - protein, 29 aa, unknown
  SMAP-29 sheep
RGLRRLGRKIAHGVKKYGPTVLRIIRIAG

SEQ ID NO: 46 - protein, 13 aa, unknown
  Indolicidine bovine
ILPWKWPWWPWRR

SEQ ID NO: 47 - protein, 18 aa, unknown
  Protegrin Porcine
RGGRLCYCRRRFCVCVGR

SEQ ID NO: 48 - protein, 31 aa, unknown
  Cecropin P1 Mammal (pig)
SWLSKTAKKLENSAKKRISEGIAIAIQGGPR

SEQ ID NO: 49 - protein, 23 aa, unknown
  Magainin frog
GIGKFLHSAKKFGKAFVGEIMNS

SEQ ID NO: 50 - protein, 25 aa, unknown
  Pleurocidin fish
GWGSFFKKAAHVGKHVGKAALTHYL

SEQ ID NO: 51 - protein, 36 aa, Aedes aegypti
GGLKKLGKKLEGAGKRVFNAAEKALPVVAGAKALRK

SEQ ID NO: 52 - protein, 40 aa, Drosophila melanogaster
GWLKKIGKKIERVGQHTRDATIQGLGIPQQAANVAATARG

SEQ ID NO: 53 - protein, 21 aa, unknown
  Buforin II vertebrate
TRSSRAGLQFPVGRVHRLLRK

SEQ ID NO: 54 - protein, 39 aa, unknown
  Sarcotoxin IA Fly
GWLKKIGKKIERVGQHTRDATIQGLGIAQQAANVAATAR

SEQ ID NO: 55 - protein, 17 aa, Apis mellifera
ANRPVYIPPPRPPHPRL

SEQ ID NO: 56 - protein, 24 aa, unknown
  Ascaphine 5 Frog
GIKDWIKGAAKKLIKTVASHIANQ

SEQ ID NO: 57 - protein, 22 aa, unknown
  Nigrocine 2 Frog
GLLSKVLGVGKKVLCGVSGLVC

SEQ ID NO: 58 - protein, 24 aa, unknown
  Pseudin 1 Rana Frog
GLNTLKKVFQGLHEAIKLINNHVQ

SEQ ID NO: 59 - protein, 18 aa, unknown
  Ranalexin Frog
FLGGLIVPAMICAVTKKC

SEQ ID NO: 60 - protein, 26 aa, unknown
  Melittin bee
GIGAVLKVLTTGLPALISWIKRKRQQ

SEQ ID NO: 61 - protein, 25 aa, unknown
  Lycotoxin 1 Spider
IWLTALKFLGKHAAKKLAKQQLSKL

SEQ ID NO: 62 - protein, 19 aa, unknown
  Parasin 1 Fish
KGRGKQGGKVRAKAKTRSS

SEQ ID NO: 63 - protein, 34 aa, Limulus polyphemus
GFKLKGMARISCLPNGQWSNFPPKCIRECAMVSS

SEQ ID NO: 64 - protein, 27 aa, artificial
  synthetic sequence
KRWVKRVKRVKRWVKRVVRVVKRWVKR

SEQ ID NO: 65 - protein, 18 aa, artificial
  synthetic sequence
GFFIPAVILPSIAFLIVP

SEQ ID NO: 66 - protein, 5 aa, artificial
  synthetic sequence
FFVAP

SEQ ID NO: 67 - protein, 39 aa, unknown
  Buforin I Toad
AGRGKQGGKVRAKAKTRSSRAGLQFPVGRVHRLLRKGNY

SEQ ID NO: 68 - protein, 13 aa, unknown
  alpha4-helix of T4 lysozyme
PNRAKRVITTFRT

SEQ ID NO: 69 - protein, 25 aa, artificial
  synthetic sequence
GKPGWLIKKALVFKKLIRRPLKRLA

SEQ ID NO: 70 - protein, 341 aa, artificial
  synthetic sequence
FFVAPKRKKRKKRKKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 71 - protein, 349 aa, artificial
  synthetic sequence
PNRAKRVITTFRTKRKKRKKRKKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 72 - protein, 354 aa, artificial
  synthetic sequence
GFFIPAVILPSIAFLIVPKRKKRKKRKKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 73 - protein, 361 aa, artificial
  synthetic sequence
GKPGWLIKKALVFKKLIRRPLKRLAKRKKRKKRKKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 74 - protein, 355 aa, artificial
  synthetic sequence
KGRGKQGGKVRAKAKTRSSKRKKRKKRKKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 75 - protein, 361 aa, artificial
  synthetic sequence
IWLTALKFLGKHAAKKLAKQQLSKLKRKKRKKRKKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 76 - protein, 341 aa, artificial
  synthetic sequence
KRKKRKKRKFFVAPKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 77 - protein, 349 aa, artificial
  synthetic sequence
KRKKRKKRKPNRAKRVITTFRTKNSEKNASIIMSIQRTLASLSLYGGRIDGLFGEKCRGAIILMLNKVYPNFSTNKLPSNTYEAESVFTFLQTALAGVGLYTITIDGKWGGTSQGAIDALVKSYRQITEAERAGSTLPLGLATVMSKHMSIEQLRAMLPTDRQGYAEVYIDPLNETMDIFEINTPLRIAHFMAQILHETACFKYTEELASGKAYEGRADLGNTRPGDGPLFKGRGLLQITGRLNYVKCQVYLREKLKDPTFDITSSVTCAQQLSESPLLAALASGYFWRFIKPKLNETADKDDIYWVSVYVNGYAKQANPYYPNRDKEPNHMKERVQMLAVTKKAHGIV

SEQ ID NO: 78 - protein, 39 aa, unknown; identical to SEQ ID NO: 67
  Buforin I Toad

SEQ ID NO: 79 - protein, 34 aa, unknown
  Dermaseptin 1 Frog
ALWKTMLKKLGTMALHAGKAALGAAADTISQGTQ

SEQ ID NO: 80 - protein, 12 aa, unknown
  Bactenecin 1 Cow
RLCRIVVIRVCR

SEQ ID NO: 81 - protein, 21 aa, unknown
  Thanatin Insect
GSKKPVPIIYCNRRTGKCQRM

SEQ ID NO: 82 - protein, 19 aa, unknown
  Brevinin 1T Rana frogs
VNPIILGVLPKVCLITKKC

SEQ ID NO: 83 - protein, 26 aa, unknown
  Ranateurin 1 Rana frog
SMLSVLKNLGKVGLGFVACKINIKQC

SEQ ID NO: 84 - protein, 46 aa, unknown
  Esculentin 1 Rana frogs
GIFSKLGRKKIKNLLISGLKNVGKEVGMDVVRTGIKIAGCKIKGEC

SEQ ID NO: 85 - protein, 17 aa, Limulus polyphemus
RWCFRVCYRGICYRKCR

SEQ ID NO: 86 - protein, 25 aa, unknown
  Androctonin Scorpion
RSVCRQIKICRRRGGCYYKCTNRPY

SEQ ID NO: 87 - protein, 30 aa, Homo sapiens
DCYCRIPACIAGERRYGTCIYQGRLWAFCC

SEQ ID NO: 88 - protein, 38 aa, unknown
  beta-defensin cow
NPVSCVRNKGICVPIRCPGSMKQIGTCVGRAVKCCRKK

SEQ ID NO: 89 - protein, 18 aa, unknown
  theta-defensin monkey
GFCRCLCRRGVCRCICTR

SEQ ID NO: 90 - protein, 40 aa, unknown
  defensin (sapecin A) insect
ATCDLLSGTGINHSACAAHCLLRGNRGGYCNGKAVCVCRN

SEQ ID NO: 91 - protein, 46 aa, unknown
  Thionin (crambin) plant
TTCCPSIVARSNFNVCRIPGTPEAICATYTGCIIIPGATCPGDYAN

SEQ ID NO: 92 - protein, 50 aa, unknown
  defensin from radish
QKLCQRPSGTWSGVCGNNNACKNQCIRLEKARHGSCNYVFPAHCICYFPC

SEQ ID NO: 93 - protein, 44 aa, Drosophila melanogaster
DCLSGRYKGPCAVWDNETCRRVCKEEGRSSGHCSPSLKCWCEGC

SEQ ID NO: 94 - protein, 25 aa, Homo sapiens
DTHFPICIFCCGCCHRSKCGMCCKT

SEQ ID NO: 95 - protein, 44 aa, unknown
  Bac 5 Cow
RFRPPIRRPPIRPPFYPPFRPPIRPPIFPPIRPPFRPPLGRPFP

SEQ ID NO: 96 - protein, 39 aa, unknown
  PR-39 Pig
RRRPRPPYLPRPRPPPFFPPRLPPRIPPGFPPRFPPRFP

SEQ ID NO: 97 - protein, 20 aa, unknown
  Pyrrhocoricin Insect
VDKGSYLPRPTPPRPIYNRN

SEQ ID NO: 98 - protein, 24 aa, Homo sapiens
DSHAKRHHGYKRKFHEKHHSHRGY

SEQ ID NO: 99 - DNA, 39 bp, artificial
  synthetic sequence
ttggaatggggagcccgaaccgtgcaaaacgtgtaatca

SEQ ID NO: 100 - DNA, 39 bp, artificial
  synthetic sequence
gtttggaggagccggtacggaaggtggtgattacacgtt

SEQ ID NO: 101 - DNA, 39 bp, artificial
  synthetic sequence
ttatgggcttcttcatcccggcagtaatcctgccctcca

SEQ ID NO: 102 - DNA, 46 bp, artificial
  synthetic sequence
gtttggaggagcccggtacgatcaggaatgcgatggagggcaggat

SEQ ID NO: 103 - DNA, 47 bp, artificial
  synthetic sequence
ttatgggcaaaccgggctggctgatcaaaaggcactggtattcaaga

SEQ ID NO: 104 - DNA, 57 bp, artificial
  synthetic sequence
gtttggaggagcctgccagtctcttcagcggacgacggatcagtttcttgaatacca

SEQ ID NO: 105 - DNA, 55 bp, artificial
  synthetic sequence
ggaatggggagcatctggctgaccgcactgaaattcctcggcaaacacgccgcaa

SEQ ID NO: 106 - DNA, 57 bp, artificial
  synthetic sequence
gtttggaggagcccagtttggataattgctgttttgccagtttctttgcggcgtgtt

SEQ ID NO: 107 - DNA, 48 bp, artificial
  synthetic sequence
ttggaatggggagcaaaggccgtggcaagcagggaggcaaagtacgtg

SEQ ID NO: 108 - DNA, 48 bp, artificial
  synthetic sequence
gtttggaggagcctgaggaacgggtctttgcttttgcacgtactttgc

SEQ ID NO: 109 - DNA, 39 bp, artificial; identical to SEQ ID NO: 99
  synthetic sequence

SEQ ID NO: 110 - DNA, 42 bp, artificial
  synthetic sequence
tatttttggaggagccggtacggaaggtggtgattacacgtt

SEQ ID NO: 111 - DNA, 39 bp, artificial; identical to SEQ ID NO: 101
  synthetic sequence

SEQ ID NO: 112 - DNA, 53 bp, artificial
  synthetic sequence
tatttttggatctgccgcccggtacgatcaggaatgcgatggagggcaggatt

SEQ ID NO: 113 - DNA, 47 bp, artificial; identical to SEQ ID NO: 103
  synthetic sequence

SEQ ID NO: 114 - DNA, 64 bp, artificial
  synthetic sequence
tatttttggatctgccgcctgccagtctcttcagcggacgacggatcagtttcttgaataccag

SEQ ID NO: 115 - DNA, 55 bp, artificial; identical to SEQ ID NO: 105
  synthetic sequence

SEQ ID NO: 116 - DNA, 60 bp, artificial
  synthetic sequence
tatttttggaggagcccagtttggataattgctgttttgccagtttctttgcggcgtgtt

SEQ ID NO: 117 - DNA, 48 bp, artificial; identical to SEQ ID NO: 107
  synthetic sequence

SEQ ID NO: 118 - DNA, 51 bp, artificial
  synthetic sequence
tatttttggaggagcctgaggaacgggtctttgcttttgcacgtactttgc

SEQ ID NO: 119 - DNA, 60 bp, artificial
  synthetic sequence
ggaatggggagctcctccaaacgcaagaaacgtaagaaacgcaaaaaaaatagcgagaat

SEQ ID NO: 120 - DNA, 24 bp, artificial
  synthetic sequence
aactattccgagtgctttctttgt

SEQ ID NO: 121 - DNA, 57 bp, artificial
  synthetic sequence
atgggatccaaacgcaagaaacgtaagaaacgcaaaggctcctccccgaaccgtgca

SEQ ID NO: 122 - DNA, 57 bp, artificial
  synthetic sequence
atgggatccaaacgcaagaaacgtaagaaacgcaaagcctcctccttcttcgtagca

SEQ ID NO: 123 - DNA, 33 bp, artificial
  synthetic sequence
ggaatggggagctcctccaaaaatagcgagaag

SEQ ID NO: 124 - DNA, 24 bp, artificial
  synthetic sequence
aactattccgtgtgctttctttgt

SEQ ID NO: 125 - DNA, 48 bp, artificial
  synthetic sequence
atgggatccttcttcgtagcaccgggctcctccaaaaatagcgagaag

SEQ ID NO: 126 - DNA, 24 bp, artificial
  synthetic sequence
aactattccgtgtgctttctttgt

SEQ ID NO: 127 - protein, 298 aa, artificial
  synthetic sequence
MKRKKRKKRKRGLRRLGRKIAHGVKKYGPTVLRIIRIAGKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 128 - protein, 622 aa, artificial
  synthetic sequence
MRGLRRLGRKIAHGVKKYGPTVLRIIRIAGKRKKRKKRKNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGLDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISYMNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGLDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISY

SEQ ID NO: 129 - protein, 318 aa, artificial
  synthetic sequence
MRGLRRLGRKIAHGVKKYGPTVLRIIRIAGRGLRRLGRKIAHGVKKYGPTVLRIIRIAGKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRK

SEQ ID NO: 130 - protein, 274 aa, artificial [sequence omitted]

SEQ ID NO: 131 - protein, 313 aa, artificial
  synthetic sequence
MGWLKKIGKKIERVGQHTRDATIQGLGIAQQAANVAATARRGLRRLGRKIAHGVKKYGPTVLRIIRIAGAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 132 - protein, 313 aa, artificial
  synthetic sequence
MAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGWLKKIGKKIERVGQHTRDATIQGLGIAQQAANVAATARRGLRRLGRKIAHGVKKYGPTVLRIIRIAG

SEQ ID NO: 133 - protein, 263 aa, artificial
  synthetic sequence
MGIGAVLKVLTTGLPALISWIKRKRQQRGLRRLGRKIAHGVKKYGPTVLRIIRIAGAISKKKVGGVGGVIAAIIAAVFAVEGGYVNDPKDPGGETNHGVTIQVAQKHKQELESMYNWDGSMKNLTQEMASSIYYNDYILKPGFVKFADVSPAVTEKLVDAGVNTGPARPSRWLQESLNAFSRNGKDYPKIQVDGKVGSGTLSAYKSLQNKRGKVEACKLILKSLDGKQLNYYLSLNMPEYTTGWIANRIGNVPLERCNEDIVN

SEQ ID NO: 134 - protein, 400 aa, artificial [sequence omitted]

SEQ ID NO: 135 - protein, 385 aa, artificial
  synthetic sequence
MGIGKFLHSAKKFGKAFVGEIMNSTRSSRAGLQFPVGRVHRLLRKVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKK

SEQ ID NO: 136 - protein, 243 aa, artificial [sequence omitted]

SEQ ID NO: 137 - protein, 232 aa, artificial
  synthetic sequence
MGIGAVLKVLTTGLPALISWIKRKRQQRGLRRLGRKIAHGVKKYGPTVLRIIRIAGNPIIDGIIALEGGYVFNPKDKGGATHWGITEATARAHGYAGDMRDLTHAEAYAILEEDYWIKPGFDVISTLSWPVSFELCDAAVNIGAYHPSAWLQRWLNVFNHEGKRYPDIHVDGNIGPRTLAALEHYLAWRGQEGEAVLVKALNCSQGTYYLNVAEKNHNNEQFIYGWIKNRVT

SEQ ID NO: 138 - protein, 376 aa, artificial
  synthetic sequence
MILPWKWPWWPWRRGLNTLKKVFQGLHEAIKLINNHVQVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVA

SEQ ID NO: 139 - protein, 549 aa, artificial
  synthetic sequence
MAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKKRKKRKKRKKRKKRKKRKGGLKKLGKKLEGAGKRVFNAAEKALPVVAGAKALRK

SEQ ID NO: 140 - protein, 298 aa, artificial
  synthetic peptide
MKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKRGLRRLGRKIAHGVKKYGPTVLRIIRIAGKRKKRKKRK

SEQ ID NO: 141 - protein, 329 aa, artificial
  synthetic sequence
MNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISYKRKKRKKRKRGLRRLGRKIAHGVKKYGPTVLRIIRIAG

SEQ ID NO: 142 - protein, 243 aa, artificial [sequence omitted]

SEQ ID NO: 143 - protein, 232 aa, artificial
  synthetic peptide
MNPIIDGIIALEGGYVFNPKDKGGATHWGITEATARAHGYAGDMRDLTHAEAYAILEEDYWIKPGFDVISTLSWPVSFELCDAAVNIGAYHPSAWLQRWLNVFNHEGKRYPDIHVDGNIGPRTLAALEHYLAWRGQEGEAVLVKALNCSQGTYYLNVAEKNHNNEQFIYGWIKNRVTRGLRRLGRKIAHGVKKYGPTVLRIIRIAGGIGAVLKVLTTGLPALISWIKRKRQQ

SEQ ID NO: 144 - protein, 313 aa, artificial
  synthetic sequence
MAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKRGLRRLGRKIAHGVKKYGPTVLRIIRIAGGWLKKIGKKIERVGQHTRDATIQGLGIAQQAANVAATAR

SEQ ID NO: 145 - protein, 376 aa, artificial
  synthetic sequence
MVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVAGLNTLKKVFQGLHEAIKLINNHVQILPWKWPWWPWRR

SEQ ID NO: 146 - protein, 385 aa, artificial
  synthetic sequence
MVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKKTRSSRAGLQFPVGRVHRLLRKGIGKFLHSAKKFGKAFVGEIMNS

SEQ ID NO: 147 - protein, 549 aa, artificial
  synthetic sequence
MKRKKRKKRKKRKKRKKRKGGLKKLGKKLEGAGKRVFNAAEKALPVVAGAKALRKAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 148 - protein, 269 aa, artificial
  synthetic sequence
MKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKKRKKRKKRK

SEQ ID NO: 149 - protein, 289 aa, artificial
  synthetic sequence
MKVLRKGDRGDEVCQLQTLLNLCGYDVGKPDGIFGNNTFNQVVKFQKDNCLDSDGIVGKNTWAELFSKYSPPIPYKTIPMPTANKSRAAATPVMNAVENATGVRSQLLLTFASIESAFDYEIKAKTSSATGWFQFLTGTWKTMIENYGMKYGVLTDPTGALRKDPRISALMGAELIKENMNILRPVLKREPTDTDLYLAHFFGPGAARRFLTTGQNELAATHFPKEAQANPSIFYNKDGSPKTIQEVYNLMDGKVAAHRKRGLRRLGRKIAHGVKKYGPTVLRIIRIAG

SEQ ID NO: 150 - protein, 320 aa, artificial
  synthetic sequence
MNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISYRGLRRLGRKIAHGVKKYGPTVLRIIRIAG

SEQ ID NO: 151 - protein, 300 aa, artificial
  synthetic sequence
MNFRTKNGYRDLQALVKELGLYTGQIDGVWGKGTSSSTETLLRGYAEVVGKNTGGIGLPTTSDASGYNVITALQRNLAFLGLYSLTVDGIWGNGTLSGDKAFEVYKERYRTPTYDIAWSGKVSPAFTAKVKDWCGVHVPNHRAPHWLMACMAFETGQTFSPSIKNAAGSEAYGLIQFMSPAANDLNVPLSVIRSMDQLTQLDLVFKYFEMWMKRGKRYTQLEDFYLTIFHPASVGKKADEVLFLQGSKAYLQNKGFDVDKDGKITLGEISSTLYTTYYKGLLPENRHVISYKRKKRKKRK

SEQ ID NO: 152 - protein, 206 aa, artificial
  synthetic sequence
MNPIIDGIIALEGGYVFNPKDKGGATHWGITEATARAHGYAGDMRDLTHAEAYAILEEDYWIKPGFDVISTLSWPVSFELCDAAVNIGAYHPSAWLQRWLNVFNHEGKRYPDIHVDGNIGPRTLAALEHYLAWRGQEGEAVLVKALNCSQGTYYLNVAEKNHNNEQFIYGWIKNRVTRGLRRLGRKIAHGVKKYGPTVLRIIRIAG

SEQ ID NO: 153 - protein, 214 aa, artificial
  synthetic sequence
MNPIIDGIIALEGGYVFNPKDKGGATHWGITEATARAHGYAGDMRDLTHAEAYAILEEDYWIKPGFDVISTLSWPVSFELCDAAVNIGAYHPSAWLQRWLNVFNHEGKRYPDIHVDGNIGPRTLAALEHYLAWRGQEGEAVLVKALNCSQGTYYLNVAEKNHNNEQFIYGWIKNRVTLLGDFFRKSKEKIGKEFKRIVQRIKDFLRNLVPRTES

SEQ ID NO: 154 - protein, 203 aa, artificial
  synthetic sequence
MNPIIDGIIALEGGYVFNPKDKGGATHWGITEATARAHGYAGDMRDLTHAEAYAILEEDYWIKPGFDVISTLSWPVSFELCDAAVNIGAYHPSAWLQRWLNVFNHEGKRYPDIHVDGNIGPRTLAALEHYLAWRGQEGEAVLVKALNCSQGTYYLNVAEKNHNNEQFIYGWIKNRVTGIGAVLKVLTTGLPALISWIKRKRQQ

SEQ ID NO: 155 - protein, 284 aa, artificial
  synthetic sequence
MAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKGWLKKIGKKIERVGQHTRDATIQGLGIAQQAANVAATAR

SEQ ID NO: 156 - protein, 274 aa, artificial
  synthetic sequence
MAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIKRGLRRLGRKIAHGVKKYGPTVLRIIRIAG

SEQ ID NO: 157 - protein, 284 aa, artificial
  synthetic sequence
MGWLKKIGKKIERVGQHTRDATIQGLGIAQQAANVAATARAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 158 - protein, 274 aa, artificial
  synthetic sequence
MRGLRRLGRKIAHGVKKYGPTVLRIIRIAGAHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 159 - protein, 352 aa, artificial
  synthetic sequence
MVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVAILPWKWPWWPWRR

SEQ ID NO: 160 - protein, 363 aa, artificial
  synthetic sequence
MVKKNDLFVDVSSHNGYDITGILEQMGTTNTIIKISESTTYLNPCLSAQVEQSNPIGFYHFARFGGDVAEAEREAQFFLDNVPMQVKYLVLDYEDDPSGDAQANTNACLRFMQMIADAGYKPIYYSYKPFTHDNVDYQQILAQFPNSLWIAGYGLNDGTANFEYFPSMDGIRWWQYSSNPFDKNIVLLDDEEDDKPKTAGTWKQDSKGWWFRRNNGSFPYNKWEKIGGVWYYFDSKGYCLTSEWLKDNEKWYYLKDNGAMATGWVLVGSEWYYMDDSGAMVTGWVKYKNNWYYMTNERGNMVSNEFIKSGKGWYFMNTNGELADNPSFTKEPDGLITVAGLNTLKKVFQGLHEAIKLINNHVQ

SEQ ID NO: 161 - protein, 364 aa, artificial
  synthetic sequence
MVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKKGIGKFLHSAKKFGKAFVGEIMNS

SEQ ID NO: 162 - protein, 362 aa, artificial
  synthetic sequence
MVKYTVENKIIAGLPKGKLKGANFVIAHETANSKSTIDNEVSYMTRNWKNAFVTHFVGGGGRVVQVANVNYVSWGAGQYANSYSYAQVELCRTSNATTFKKDYEVYCQLLVDLAKKAGIPITLDSGSKTSDKGIKSHKWVADKLGGTTHQDPYAYLSSWGISKAQFASDLAKVSGGGNTGTAPAKPSTPAPKPSTPSTNLDKLGLVDYMNAKKMDSSYSNRDKLAKQYGIANYSGTASQNTTLLSKIKGGAPKPSTPAPKPSTSTAKKIYFPPNKGNWSVYPTNKAPVKANAIGAINPTKFGGLTYTIQKDRGNGVYEIQTDQFGRVQVYGAPSTGAVIKKTRSSRAGLQFPVGRVHRLLRK

SEQ ID NO: 163 - protein, 513 aa, artificial
  synthetic sequence
MKRKKRKKRKKRKKRKKRKAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK

SEQ ID NO: 164 - protein, 531 aa, artificial
  synthetic sequence
MGGLKKLGKKLEGAGKRVFNAAEKALPVVAGAKALRKAKTQAEINKRLDAYAKGTVDSPYRVKKATSYDPSFGVMEAGAIDADGYYHAQCQDLITDYVLWLTDNKVRTWGNAKDQIKQSYGTGFKIHENKPSTVPKKGWIAVFTSGSYEQWGHIGIVYDGGNTSTFTILEQNWNGYANKKPTKRVDNYYGLTHFIEIPVKAGTTVKKETAKKSASKTPAPKKKATLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTVVKDGKTSSASTPATRPVTGSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFK